(19) United States
(12) Patent Application Publication
(10) Pub. No.: US 2021/0378572 A1
(43) Pub. Date: Dec. 9, 2021

(54) INDUCTOR-CAPACITOR BASED SENSOR APPARATUSES

(71) Applicant: The Board of Trustees of the Leland Stanford Juior University, Stanford, CA (US)

(72) Inventors: Levent Beker, Stanford, CA (US); Clementine M. Boutry, Stanford, CA (US); Zhenan Bao, Stanford, CA (US)

(21) Appl. No.: 17/287,805

(22) PCT Filed: Oct. 25, 2019
(86) PCT No.: PCT/US2019/058192

§ 371 Date: Apr. 22, 2021

Related U.S. Application Data
(60) Provisional application No. 62/750,518, filed on Oct. 25, 2018.

(51) Int. Cl. A61B 5/277; A61B 5/021

(52) U.S. Cl. CPC A61B 5/277; A61B 2017/1107; A61B 5/021

(57) ABSTRACT

An example sensor apparatus includes two inductors with a first elastomer material between and at least one capacitor coupled to the two inductors. The at least one capacitor is configured, while in use, to at least partially wrap a circumference of an object and to exhibit a change in impedance in response to a pressure-manifestation change associated with the object, the change in impedance is to cause a change in the resonant frequency of the two inductors.

### OVERVIEW

In clinical environments, various types of physiological sensors and related methods are used. One type is an implant for monitoring blood vessels. In this context, vessel anastomosis (the surgical technique used to make a connection between blood vessels) is one of the important procedures in cardiovascular, vascular, and transplantation surgeries. Often failure is not identified until the opportunity to save the graft has passed, making repeat intervention required. It has been shown that surgical salvage is closely correlated with the intensity of monitoring and time from detection to return to the operating room. Other existing methods available in clinical environments are either non precise (visual assessment of skin color and turgor), or require expensive equipment only available in a hospital (external Doppler evaluation). A clinically available device to address this problem for microsurgical anastomoses is an implantable Doppler system where an implantable ultrasonic probe is mounted on the vessel. However, this uses wired connections limiting its use to the hospital environment. Moreover, the device must be removed after use.

Similarly, a cuff-type Hall-effect sensor for pulse-monitoring may be used. This sensor includes silicon chips and magnets to be placed over the patient's targeted cardiovascular-related anatomy and as such it requires surgical removal. Other reported methods include color duplex sonography, near infrared spectroscopy, microdialysis, and laser Doppler flowmetry. Even though several of these methods can provide an accurate and preventative insight, they are costly and complex, requiring highly trained clinicians for operation.

The above issues as well as others have presented challenges to sensor apparatuses for a variety of applications.

### SUMMARY

Aspects of various embodiments are directed to a sensor apparatus that includes an inductor and capacitor for detecting changes in pressure or forces based on resonant frequency shifts.

In certain example embodiments, aspects of the present disclosure involve a sensor apparatus that senses forces applied thereto based on a change in resonant frequency of an inductor of the apparatus that is caused by a change in impedance of a capacitor of the apparatus. The inductor and capacitor may be arranged such that a wireless link to sensing circuitry is decoupled from the sensing of the force applied to the sensor apparatus.

More specific aspects of the present disclosure are directed to physiological sensors including circuitry and materials configured to be adjacent or affixed to a living being and to detect and report (e.g., indicate) cardiovascular-related attributes associated with a set of one or more physiological conditions.

A specific embodiment is directed to a sensor apparatus that includes two inductors and at least one capacitor. The two inductors have a first elastomer material between, and are coupled to the at least one capacitor. The at least one capacitor is configured, while in use, to at least partially wrap a circumference of an object and to exhibit a change in impedance in response to a pressure-manifestation change associated with the object, the change in impedance is to cause a change in the resonant frequency of the two inductors. The pressure-manifestation change may cause a force applied to the at least one capacitor, such as pressure or a change in electromagnetic field. The two inductors and the at least one capacitor may include an inductance-capacitance-resistance (LCR) resonator circuit formed by first and second wires, and the two inductors are formed of portions of the first and second wires as respectively arranged in a coil. As such, the two inductors, the first and second wires and the at least one capacitor are integrated together and may be formed of the same material.

In various examples, the two inductors with the first elastomer material provide a wireless link to a reader coil concurrently with the least one capacitor exhibiting the change in impedance and causing the change in resonant frequency. The wireless link provided may be independent (e.g., is decoupled) from sensing of the pressure-manifestation change.

In a number of embodiments, the at least one capacitor includes portions of first and second wires coupled to the two inductors. The portions of the first and second wires may form first and second electrodes of the capacitor. A dielectric material may expand the portions of the first and second wires. The dielectric material, in specific embodiments, includes a structured dielectric material that overlaps the portions of the first and second wires. For example, the at least one capacitor includes a fringe-field capacitor and the apparatus is biodegradable, and the sensor apparatus is configured to respond to pressure applied thereto in a contact mode and to respond to a change in electromagnetic field in a non-contact mode. The apparatus may further include a reader coil and circuitry coupled to the reader coil to detect the change in the resonant frequency and to determine the pressure-manifestation change based on the change in the resonant frequency.

In various specific embodiments, the at least one capacitor includes a first capacitor and a second capacitor formed of portions of a first wire and a second wire. The first capacitor includes a first portion of the first wire forming a first electrode and a first portion of the second wire forming a second electrode. The second capacitor includes a second portion of the first wire forming a third electrode and a second portion of the second wire forming a fourth electrode. The first and second capacitors may further include a dielectric material including a first dielectric material expanding the first portions of the first and second wires and a second dielectric material expanding the second portions of the first and second wires.

In other example embodiments, a sensor apparatus includes a first inductive coil and a second inductive coil with a first elastomer material between, and a first wire coupled to the first inductive coil and a second wire coupled to the second inductive coil. The sensor apparatus further includes a first capacitor. The first capacitor includes a first portion of the first wire and a first portion of the second wire, and a first dielectric material that expands between the first portions of the first and second wires. In specific embodiments, the sensor apparatus further includes a second capacitor including a second portion of the first wire and a second portion of the second wire, and a second dielectric material that expands between the second portions of the first and second wires. The first and, optionally, second capacitors are configured to, while in use, at least partially wrap a circumference of an object and to exhibit a change in impedance in response to a pressure-manifestation change associated with the object, and the change in impedance is to cause a change in the resonant frequency of the two inductive coils. For example, the first and second capacitors are configured to wrap around a circular vessel and to exhibit the change in impedance in response to a force applied (e.g., pressure or changes in electromagnetic field) by the circular vessel. In other examples and/or in addition, the first and/or second capacitors include fringe-field capacitors that are configured to wrap around an artery of a user and to exhibit the change in impedance in response to pressure applied or a change in electromagnetic field caused by the artery, and the sensor apparatus is biodegradable.

In a number of related embodiments, the first and/or second dielectric materials include a substrate with embedded three-dimensional (3D) microstructures. For example, the 3D microstructures include pyramid-shaped microstructures. Additionally or alternatively, the apparatus further includes a second elastomer material proximal to one of the first and second inductive coils and the dielectric material (e.g., the first dielectric material and optionally, the second dielectric material), and a third elastomer material proximal to the second of the first and second inductive coils.

Other embodiments are directed to methods of forming the above-described sensor apparatuses. An example method includes forming a first inductive coil coupled to a first wire and a second inductive coil coupled to a second wire from a conductive material, and forming a first elastomer material on one of the first and second inductive coils. Forming the first and second inductive coils, the first wire, and the second wire may include laser cutting the first inductive coil coupled to the first wire and the second inductive coil coupled to the second wire from the conductive material, the first and second inductive coils being coupled together. The method further includes aligning the first conductive coil and the second conductive coil such that the first elastomer material is there between and the first and second wires extend from the first and second inductive coils at a first end of the first and second wires with a distance between at a second end of the first and second wires. Aligning the first and second inductive coils may include folding the second inductive coil to align with the first inductive coil. And, the method includes forming a dielectric material that expands a portion of the first and second wires proximal to the second ends of the first and second wires, wherein the portion of the first and second wires form at least one capacitor of the sensor apparatus. The at least one capacitor is configured to, while in use, at least partially wrap a circumference of an object and to exhibit a change in impedance in response to a pressure-manifestation change associated with the object, and the change in impedance is to cause a change in the resonant frequency of the first and second inductive coils.

In a number of embodiments, the method further includes laminating the sensor apparatus with a second elastomer material proximal to one of the first and second inductive coils and the dielectric material, and a third elastomer material proximal to the second of the first and second inductive coils. The first and second inductive coils form an antenna and the at least one capacitor forms a sensing region of the sensor apparatus. Additionally, the formed sensor apparatus is biodegradable and implantable.

For addressing such above-noted issues as well as providing other advantages, various example embodiments disclosed herein are directed to apparatuses, systems, methods of use, methods of making, or materials, such as those described in the claims, descriptions or figures herein and in the attached Appendix entitled “Wireless Monitoring of Blood Flow via Biodegradable, Flexible, Passive Arterial Pulse Sensor” (and the second or supplemental Appendix entitled “Structural and electrical effect of planar double capacitor design”) all of which form part of this patent document. For information regarding details of other embodiments, experiments and applications that can be combined in varying degrees with the teachings herein, reference may be made to the teachings and underlying references provided in and by way of one or both of the included/attached appendices which form a part of this patent document and are fully incorporated herein by reference.

Various aspects, including but not limited to those in the attached appendices, are directed toward an apparatus and related methodology based on a device that monitors blood flow by way of its construction which provides for various advantageous attributes including one or more of the following which facilitates convenience, use and implantability, among other advantages: biodegradable materials which offsets the need for post-implant surgery, flexibility (e.g., specifically with regards to adapting the sensing device to the target region being monitored), wireless operation (e.g., via a battery-free sensor (circuit)).

Accordingly, various embodiments are directed to addressing challenges relating to the above aspects, and others, as may benefit by varying such features and materials as needed for a particular cardiovascular application. For instance, certain embodiments are directed to aspects and features brought out in the provisional claims, figures and embodiments disclosed herein (including the Appendix entitled “Wireless Monitoring of Blood Flow via Biodegradable, Flexible, Passive Arterial Pulse Sensor” and also the second or supplemental Appendix entitled “Structural and electrical effect of planar double capacitor design”).

The above discussion/summary is not intended to describe each embodiment or every implementation of the present disclosure. The figures and detailed description (and referring the underlying Provisional Application and the appendixes are fully incorporated herein) that follow also exemplify various embodiments.

### BRIEF DESCRIPTION OF FIGURES

Various example embodiments may be more completely understood in consideration of the following detailed description in connection with the accompanying drawings, in which:

FIGS. 1A-1C show example sensor apparatuses, consistent with embodiments of the present disclosure;

FIGS. 2A-2H show an example sensor apparatus having different capacitor arrangements and resulting pressure detection, consistent with embodiments of the present disclosure;

FIGS. 3A-3E show example inductive arrangements and forming of the same of a sensor apparatus, consistent with embodiments of the present disclosure;

FIGS. 4A-4G show example capacitive arrangements of a sensor apparatus, consistent with embodiments of the present disclosure;

FIGS. 5A-5G show example inductive arrangements of sensor apparatus and resulting resonant frequency shifts, consistent with embodiments of the present disclosure;

FIGS. 6A-6F show an example of a sensor apparatus under different forces, consistent with embodiments of the present disclosure;

FIGS. 7A-7G show another example sensor apparatus and responses to pressure applied, consistent with embodiments of the present disclosure;

FIGS. 8A-8H show example results of an implanted sensor apparatus under different forces and resulting responses, consistent with embodiments of the present disclosure;

FIGS. 9A-9B show example performance of two different sensor apparatuses, consistent with embodiments of the present disclosure;

FIGS. 10A-10B show an example of an implanted sensor apparatus, consistent with embodiments of the present disclosure;

FIGS. 11A-11E show examples of an implanted sensor apparatus, consistent with embodiments of the present disclosure;

FIGS. 12A-12B show example resonant frequency shifts of a sensor apparatus, consistent with embodiments of the present disclosure;

FIGS. 13A-13B show further example capacitance changes of a sensor apparatus, consistent with embodiments of the present disclosure;

FIG. 14 shows example sensitivity of a sensor apparatus, consistent with embodiments of the present disclosure; and

FIG. 15 shows example force characterized by a sensor apparatus, consistent with embodiments of the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are applicable to a variety of different types of apparatuses and methods involving a sensor apparatus that senses forces applied thereto based on a change in resonant frequency of an inductor of the apparatus that is caused by a change in impedance of a capacitor of the apparatus. In a number of implementations, the inductor and capacitor may be arranged such that a wireless link to sensing circuitry is decoupled from sensing of the force applied. In certain implementations, aspects of the present disclosure have been shown to be beneficial when used in the context of biodegradable sensor apparatus for implantable sensing, such as for wireless monitoring of arterial blood flow via the sensor apparatus, but it will be appreciated that the instant disclosure is not necessarily so limited. Various aspects may be appreciated through the following discussion of non-limiting examples which use exemplary contexts.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination.

The sensor apparatus, in accordance with the present disclosure, may be used to provide wireless monitoring of a pressure or changes in an electromagnetic field applied to the sensor apparatus. The sensor may include an inductance-capacitance-resistance (LCR) resonator circuit that includes an inductive arrangement and a capacitive arrangement. The inductive arrangement and capacitive arrangement may decouple the wireless link to sensing circuitry from sensing of the force applied thereto such that the sensor apparatus may concurrently provide a wireless link and sense the applied force (e.g., pressure and/or associated with the change in electromagnetic field). The wireless link provided, e.g., reading of the resonant frequency of the inductor by a reader coil, may be independent from and/or may not impact the sensing of the applied force, and the sensing of applied force may be independent from and/or may not impact the wireless link provided. As further described herein, the sensor apparatus may be formed of flexible elastomer material and conductive wires, such that a portion of the sensor apparatus may wrap around at least a portion of an object and respond to pressure changes and/or electromagnetic field changes applied by the object. Further, the various materials may be biodegradable, such that the sensor apparatus may be implanted in an organism and may be biodegradable. As such, once implanted, the sensor apparatus may degrade and be removed from the organism without an additional surgery. In specific examples, the sensor apparatus may be used for wireless monitoring of arterial blood flow, as further described herein.

A specific embodiment is directed to a sensor apparatus that includes two inductors and at least one capacitor. The two inductors have a first elastomer material between, and are coupled to the at least one capacitor. The at least one capacitor is configured, while in use, to at least partially wrap a circumference of an object and to exhibit a change in impedance in response to a pressure-manifestation change associated with the object, the change in impedance is to cause a change in the resonant frequency of the two inductors. The two inductors and the at least one capacitor may include an LCR resonator circuit formed by a first wire and a second wire, and the two inductors are formed of portions of the first and second wires as respectively arranged in a coil. As such, the two inductors, the first and second wires and the at least one capacitor are integrated together.

In other example embodiments, a sensor apparatus includes a first inductive coil and a second inductive coil with a first elastomer material between, and a first wire coupled to a first inductive coil and a second wire coupled to the second inductive coil. The sensor apparatus further includes a first capacitor, and optionally, a second capacitor. The first capacitor includes a first portion of the first wire and a first portion of the second wire, and a first dielectric material that expands between the first portions of the first and second wires. The second capacitor includes a second portion of the first wire and a second portion of the second wire, and a second dielectric material that expands between the second portions of the first and second wires. The first capacitor (and optionally, the second capacitors) is configured to, while in use, at least partially wrap a circumference of an object and to exhibit a change in impedance in response to a pressure-manifestation change associated with the object, and the change in impedance is to cause a change in the resonant frequency of the two inductive coils. For example, the first and second capacitors are configured to wrap around a circular vessel and to exhibit the change in impedance in response to pressure applied by the circular vessel. In other examples, the first and second capacitors include fringe-field capacitors that are configured to wrap around an artery of a user and to exhibit the change in impedance in response to pressure applied or a change in electromagnetic field caused by the artery, and the sensor apparatus is biodegradable.

Other embodiments are directed to methods of forming the above-described sensor apparatuses, as further described herein.

Aspects of the present disclosure are believed to be applicable to a variety of different types of apparatuses, systems and methods involving wireless monitoring of arterial blood flow via pulse sensors and sensor circuitry. While not necessarily so limited, various aspects may be appreciated through a discussion of examples using this context and more specifically by monitoring arterial blood flow via a device that includes and/or is characterized by its primary attributes which are: biodegradable, flexible, wireless and being operable by a battery-free sensor (circuit/circuitry). Various such embodiments are also described in the attached appendices which forms part of this patent document and are incorporated by reference for their teaching.

Aspects of the instant disclosure are directed to methods, apparatuses (e.g., systems, devices and circuitry) configured for short term monitoring of arterial blood flow using a biodegradable, flexible, wireless and battery-free sensor mounted on an artery, which can be applied to various surgical operations. In one specific embodiment, a cuff-type sensor circuit has a thin and flexible structure that allows it to be easily wrapped around arteries of various sizes. The blood flow is measured using a fringe-field capacitive sensor design. Wireless operation is enabled using radio frequency (RF) inductive coupling method. Furthermore, the device can be entirely made of biodegradable materials and can be resorbed by the body after several months, eliminating the need for device removal. Sensor operation is demonstrated in vitro with an artificial artery model and in vivo in a rat model, showing excellent biocompatibility and pulse monitoring function for wired and wireless sensor configurations.

An example fringe field capacitive sensor device includes a fringe-field capacitive sensor that is sensitive both in contact and non-contact modes, and a bi-layer coil structure that is used for RF data transmission. Due to the pulsatile nature of blood flow, the artery experiences a change in vessel diameter over time that is measured by the capacitive sensor. The change in impedance results in a shift of the resonant frequency of the LCR resonator circuit, monitored wirelessly through the skin via inductive coupling with an external reader coil in a battery-free approach. The wireless circuit may include an LCR resonator (inductor-capacitor-resistor resonant circuit), wherein the capacitive sensor is connected in series with an inductor coil. The change in impedance due to artery expansion results in a shift of the resonant frequency ƒ₀ of the LCR resonator circuit. This shift is monitored wirelessly through the skin by inductive coupling with an external reader coil, on the ports of which the scattering parameter S11 is measured.

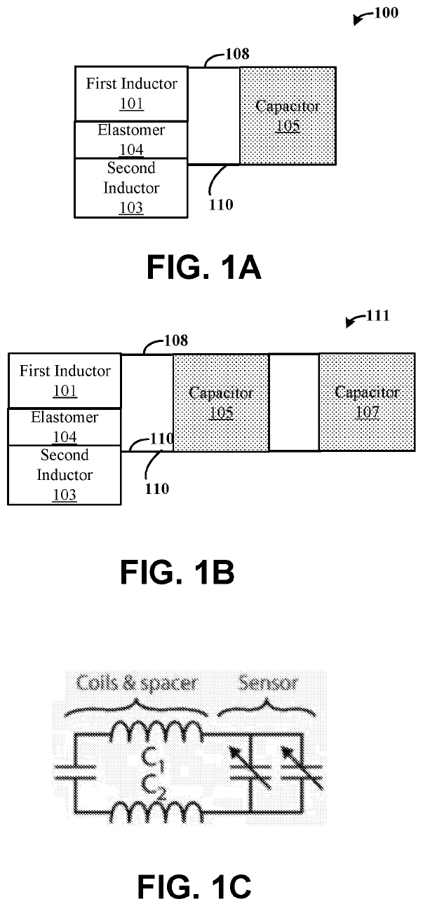

Turning now to the figures, FIGS. 1A-1C show example sensor apparatuses, consistent with embodiments of the present disclosure. As previously described, the apparatus includes an LCR resonator circuit that includes two inductors 101, 103 and at least one capacitor 105, 107. In various examples, the apparatuses 100, 111 are biodegradable.

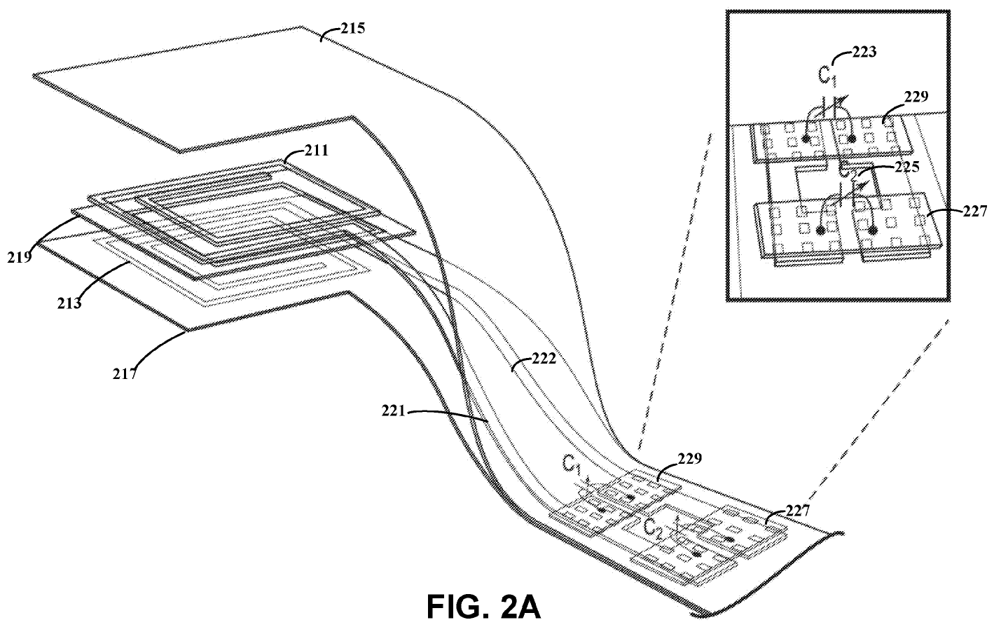

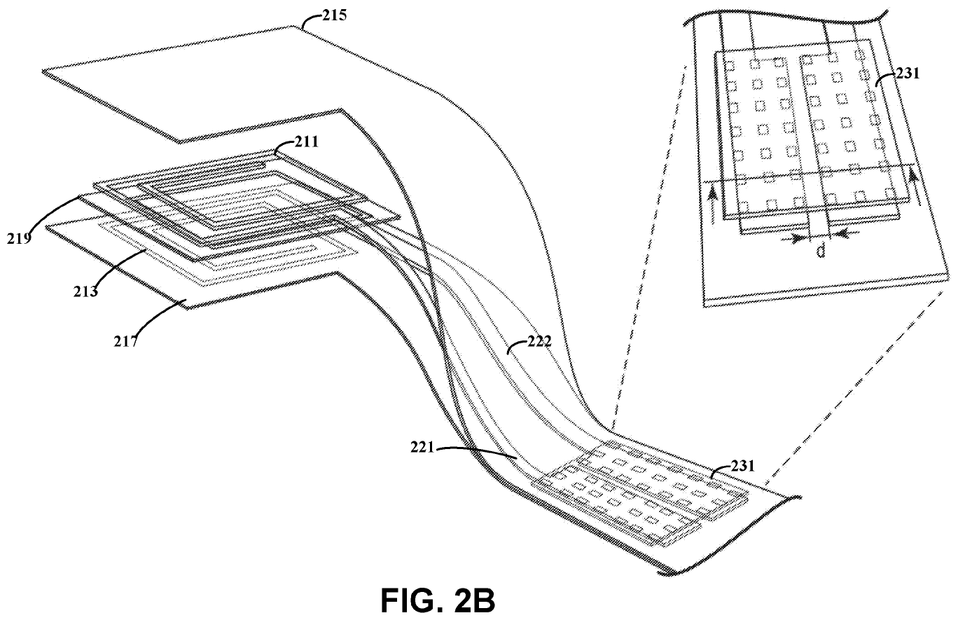

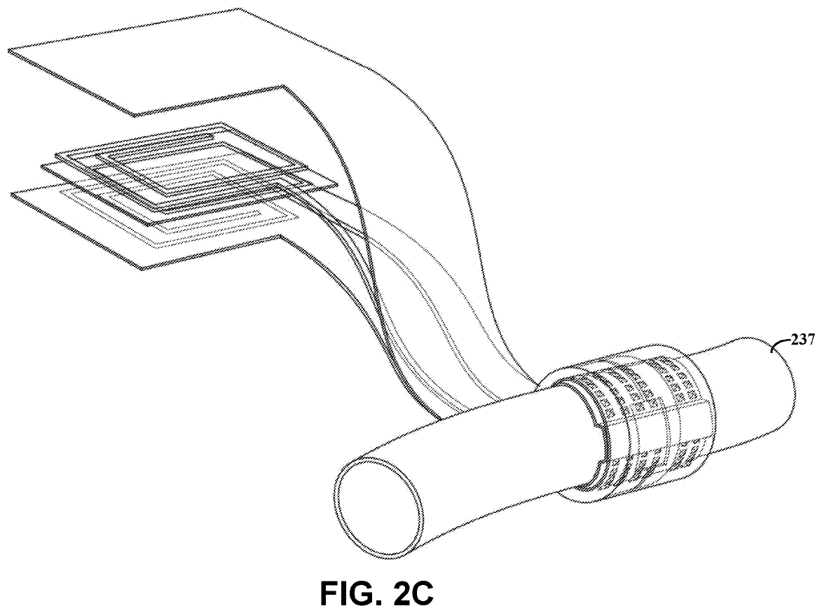

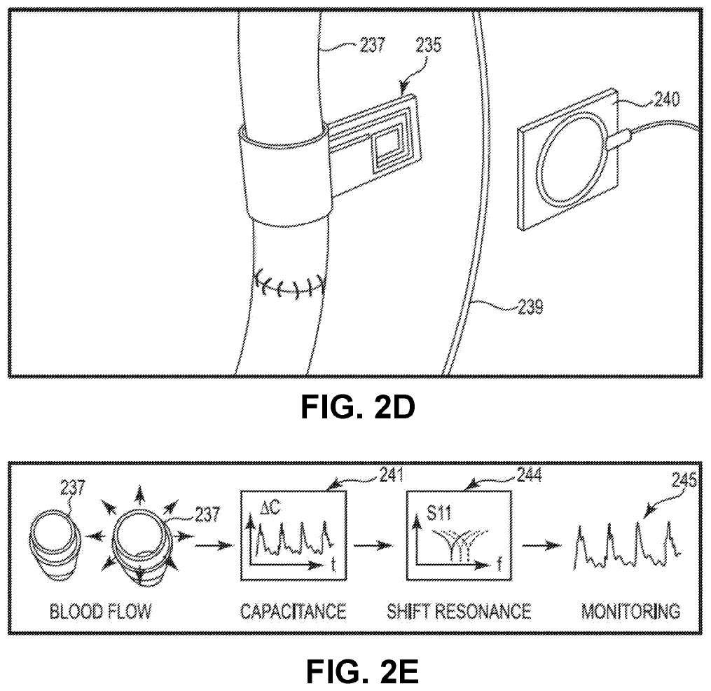

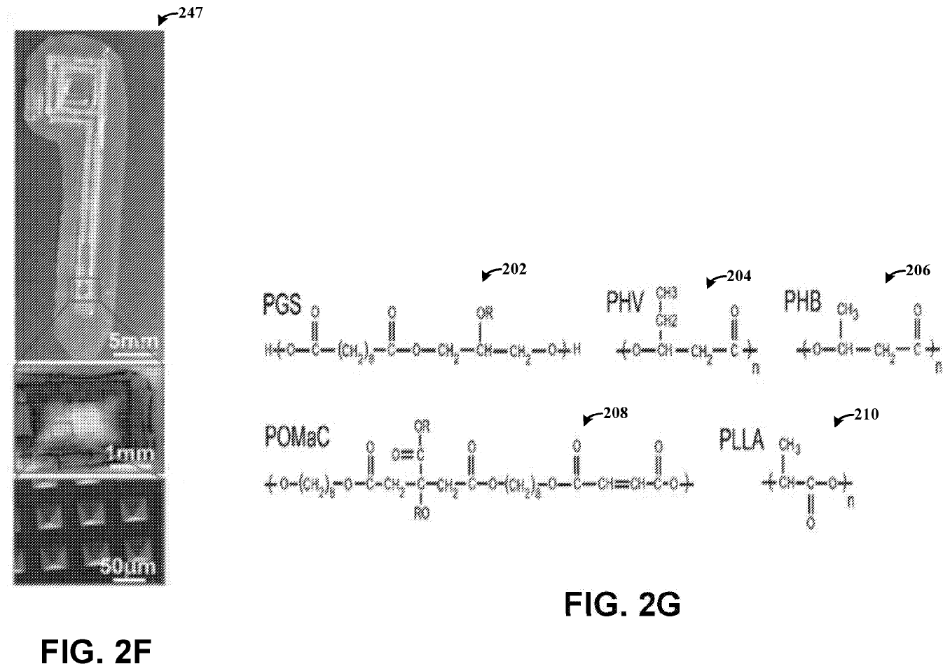

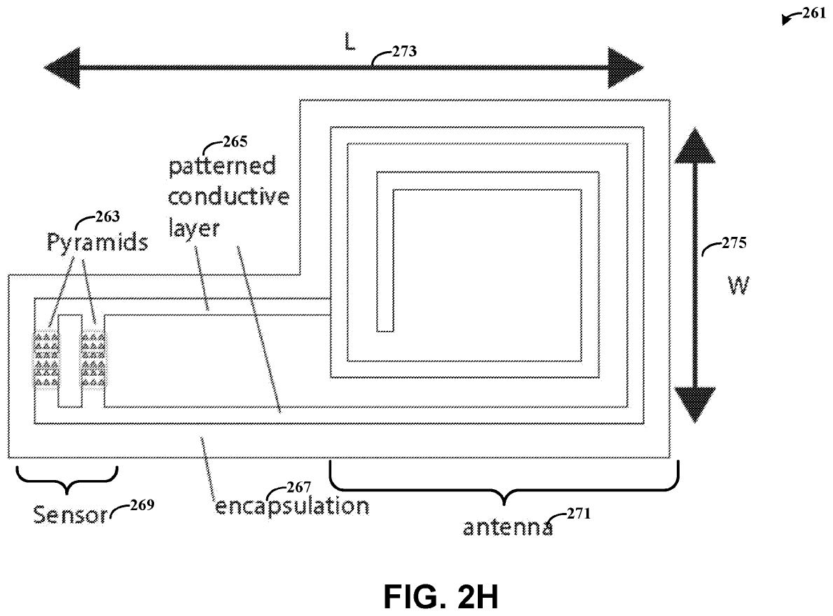

FIG. 1A illustrates an example of a sensor apparatus that includes one capacitor, which is further illustrated by FIG. 2B. As shown, the sensor apparatus 100 includes two inductors 101, 103 with a first elastomer material 104 between. The first elastomer material 104 may prevent electrical shorting between the two inductors 101, 103. In specific embodiments, each of the first and second inductors 101, 103 include inductive coils. The apparatus 100 further includes at least one capacitor 105 coupled to the two inductors 101, 103, such as being coupled via the first and second wires 108, 110. The two inductors 101, 103 may be formed of portions of the first and second wires 108, 110 as respectively arranged in a coil.

The at least one capacitor 105, and/or the two inductors 101, 103, may be formed by first and second wires 108, 110 (with portions of the wires forming each). In specific examples, the two inductors 101, 103 and the at least one capacitor 105 include an LCR resonator circuit formed by the first and second wires 108, 110. The at least one capacitor 105 may include portions of the first and second wires 108, 110 which form first and second electrodes of the capacitor 105, and a dielectric material that expands the portions of the first and second wires 108, 110. While in use, the capacitor 105 is configured to at least partially wrap a circumference of an object, such as a circular vessel. The capacitor 105 exhibits a changes in impedance (e.g., capacitance or resistance) in response to a pressure-manifestation change associated with the object, and the change in impedance causes a change in a resonant frequency of the two inductors 101, 103 (e.g., shifts the resonance). The pressure-manifestation change, as used herein, includes or refers to a change in a parameter of the object or by the object and/or which may be in response to a pressure change associated with the object. For example, the pressure-manifestation change may include a change in a diameter and/or the circumference of the object which causes a pressure applied to the capacitor 105 or a change in an electromagnetic field associated with the capacitor 105. The change in electromagnetic field sensed may include a range, such 5 megahertz (MHz) to 244 gigahertz (GHz) (e.g., not less than 5 MHz to not more than 244 GHz) and in other examples 50 MHz to 100 GHz (e.g., not less than 50 MHz and not more than 100 GHz). For example, the object may include an artery which changes circumference size in response to blood flow. As another example, the object may include a stent that is implanted in an animal, and a temperature inside the animal may cause the stent to expand or contract. As another example, the object may include a robotic or machinery that changes size in response to atmospheric or room temperatures.

As further described herein, the capacitor 105 may include a fringe-field capacitor. In specific examples, the dielectric material includes a structured dielectric material having microstructures embedded thereon and/or that overlaps the portions of the first and second wires 108, 110. The microstructures may include three-dimensional (3D) microstructures, such as pyramid-shaped microstructures.

As illustrated by FIG. 1B, sensor apparatuses are not limited to one capacitor. For example, the sensor apparatus 111 illustrated by FIG. 1B includes first and second inductors 101, 103, the first and second wires 108, 110 as previously described by FIG. 1A, and additionally includes a first capacitor 105 and a second capacitor 107. As previously described, the first and second inductors 101, 103 may include first and second inductive coils, and the first wire 108 couples to the first inductive coil 101 and the second wire 110 couples to the second inductive coil 103. The first and second capacitors 105, 107 exhibit a change in impedance in response to the pressure-manifestation change of the object, and the change in impedance is to cause a change in the resonant frequency of the two inductive coils (e.g., shifts the resonance).

The first capacitor 105 includes a first portion of the first wire 108 and a first portion of the second wire 110, and a first dielectric material that expands between the first portions of the first and second wires 108, 110. The first portion of the first wire 108 may form a first electrode and the first portion of the second wire 110 may form a second electrode, with the first and second electrodes pairing to form the first capacitor 105. The second capacitor 107 includes a second portion of the first wire 108 and a second portion of the second wire 110, and a second dielectric material that expands between the second portions of the first and second wires 108, 110. The second portion of the first wire 108 may form a third electrode and the second portion of the second wire 110 may form a fourth electrode, with the third and fourth electrodes pairing to form the second capacitor 107. The first and second capacitors 105, 107 may include fringe-field capacitors.

The first and second dielectric materials may include a structured dielectric material having microstructures embedded thereon. For example, the first and second dielectric materials each include a substrate with embedded 3D microstructures, such as pyramid-shaped microstructures. In a specific example, the first and second dielectric materials are formed of poly(glycerol sebacate (PGS) and the 3D microstructures include pyramid-shaped PGS microstructures.

Although the embodiment of FIG. 1B illustrates two capacitors, embodiments are not so limited and may include a first capacitor coupled to the two inductors, such as illustrated by FIG. 1A.

In various examples, although not illustrated, one or more of apparatuses 100, 111 further includes a second elastomer material proximal to a first of the two inductors 101, 103, and a third elastomer material proximal to the second of the two inductors 101, 103. The first, second, and third elastomer materials may include different elastomers that are each biodegradable. In specific examples, the first elastomer material includes poly(lactic acid) (PLLA), the second elastomer material includes poly(octamethylene maleate (anhydride) citrate) (POMaC), the third elastomer material includes polyhydroxybutyrate/polyhydroxyvalerate (PHB/PHV), and the (first and/or second) dielectric material of the capacitors 105, 107 may include thick poly(glycerol sebacate) (PGS). Although embodiments are not so limited, and the elastomer materials may be formed of a variety of different types of polymers, such as alginate-based supramolecular ionic polyurethanes (ASPUs), polycaprolactone (PCL), poly-4-hydroxybutyrate (P4HB), and various other hydrogels, elastin-like peptides, and polyhydroxyalkanoate (PHA).

The inductors 101, 103 may be used to wirelessly communicate with a reader coil. For example, the reader coil may be used to detect the changes in a resonant frequency of the inductors 101, 103, and is in communication with circuitry (e.g., processor) to determine the pressure-manifestation change associated with the object (e.g., pressure applied to the sensor apparatuses 100, 111 or the change in electromagnetic field) based on the change in resonant frequency. The two inductors 101, 103 with the first elastomer material 104 there between may decouple the wireless link to the reader coil from the sensing of the pressure-manifestation change. For example, the two inductors 101, 103 provide the wireless link to a reader coil currently with the at least one capacitor 105, 107 exhibiting the change in impedance and causing the change in resonant frequency. The at least one capacitor 105, 107 may exhibit the change in impedance independent from the wireless link provided.

The sensor apparatuses 100, 111 and/or portions thereof may be flexible such that the at least one capacitor 105, 107 may be wrapped around a circular vessel and exhibit the change in impedance in response to the pressure-manifestation change, such as changes in pressure applied by the circular vessel and/or changes in the electromagnetic field caused by changes in the circumferences of the circular vessel. As used herein, a circular vessel is not limited to a perfectly circular shape, and may include imperfections, such as exhibited by an artery. As a specific example, a portion of the sensor apparatus 111 that includes the first capacitor 105 and, optionally, the second capacitor 107 may wrap around (or at least partially wrap around) the circular vessel, such as an artery of a mammal. The first and second capacitors 105, 107 exhibit the change in impedance in response to pressure changes applied by the artery and/or the change in electromagnetic field, and the sensor apparatus 111 is biodegradable.

FIG. 1C illustrates the equivalent electrical circuit of the sensor apparatus 111 illustrated by FIG. 1B. The two variable capacitors correspond to C1 and C2. The two inductors in series with a fixed capacitor correspond to the top and bottom Mg coils and the PLLA insulation material, respectively.

FIGS. 2A-2H show an example sensor apparatus having different capacitor arrangements and resulting pressure detection, consistent with embodiments of the present disclosure.

FIG. 2A illustrates an example sensor apparatus having first and second capacitors 223, 225, such as that previously described in connection with FIG. 1B. As shown, the sensor apparatus includes the first and second inductive coils 211, 213 which are separated by the first elastomer material, which includes PLLA 219. First and second wires 221, 222 are coupled to the first and second inductive coils 211, 213 and first and second capacitors 223, 225. The first and second capacitors 223, 225 are formed of portions of the first and second wires 221, 222 and include the first and second dielectric material 227, 229, which include PGS pyramid-shaped microstructures. The portions of the wires 221, 222 forming the capacitors 223, 225 include ends of the first and second wires 221, 222 which are distal to the first and second inductive coils 211, 213. The sensor apparatus is laminated using a second elastomer material of POMaC 215, and a third elastomer material of PHB/PHV 217, in specific examples.

FIG. 2B illustrates an example sensor apparatus having one capacitor, such as that previously described in connection with FIG. 1A. As shown, the sensor apparatus includes the first and second inductive coils 211, 213 which are separated by the first elastomer material, which includes PLLA 219. First and second wires 221, 222 are coupled to the first and second inductive coils 211, 213 and the capacitor formed by the portions of the first and second wires 221, 222. The portions of the wires 221, 222 forming the capacitor include ends of the first and second wires 221, 222 which are distal to the first and second inductive coils 211, 213. The capacitor includes first and second electrodes that are formed of the portions of the first and second wires 221, 222 and includes the dielectric material 231 which include PGS pyramid-shaped microstructures. The sensor apparatus is laminated using a second elastomer material of POMaC 215, and a third elastomer material of PHB/PHV 217, as previously described.

In various examples, the sensor apparatus may be implanted in an organism, such as a mammal, and wrapped around an (or at least partially around) artery of the organism, as illustrated by FIG. 2C. FIG. 2C includes a sensor apparatus as previously illustrated and described in connection with FIGS. 1B and 2A. More specifically, FIG. 2C is a schematic illustration of the sensor apparatus with exposed view of the bi-layer coil structure for wireless data transmission and cuff-type pulse sensor wrapped around the artery 237. The separation between the sensor (e.g., capacitors) and the antenna (e.g., inductive coils) can be adapted to the application site. In a specific experimental embodiments, 3.2 cm-long devices are fabricated in consideration of the dimensions of the femoral artery of a rat.

Vessel anastomosis, which is the surgical technique used to make a connection between blood vessels, is one of the most critical portions of cardiovascular, vascular, and transplantation surgeries (see FIG. 1A of the underlying Provisional Application). For example, patients may undergo coronary artery bypass grafting. Other common diseases requiring revascularization include critical limb ischemia and traumatic vascular repair surgeries. Additionally, there is an expanding role for microvascular free tissue transfer to reconstruct patients after cancer or trauma. After each of these operations, ensuring blood flow through the newly created anastomosis is critical.

The sensor apparatus, in accordance with various embodiments, allows for wireless and battery-free monitoring of arterial blood flow that can be applied to various surgical operations. The sensor apparatus is cuff-like and has a thin and flexible structure that allows the device to be wrapped around arteries of various sizes. Wireless operation is enabled using various radio frequency (RF) coupling methods. Furthermore, the device may be entirely made of biodegradable materials and is resorbed after several months, eliminating a device removal procedure. As further illustrated herein, sensor operation is demonstrated in vitro with a custom-made artificial artery model and in vivo in a rat model, showing excellent biocompatibility and pulse monitoring function for wired and wireless sensor configurations. Accordingly, embodiments are directed to a biodegradable sensor able to monitor locally and in real-time the blood flow in an artery, allowing for wireless monitoring of vessel anastomoses after surgery.

FIG. 2D illustrates an example of wirelessly monitoring blood flow using a sensor apparatus 235, such as that illustrated by FIGS. 2A-2B. As a specific example, after reconstructive surgery involving microsurgical anastomosis, failure may occur via formation of a hematoma or thrombosis within the artery or vein. It has been shown that surgical salvage is closely correlated with the intensity of monitoring and time from detection to return to the operating room. The biodegradable sensor, in accordance with various embodiments, can be used to wirelessly monitor the arterial blood flow after microvascular reconstruction surgery continuously, eliminating a lag time between loss of blood flow and detection. The sensor apparatus 235 may work on the smallest blood vessels with the smallest change in diameter during pulsation. With simple reconfiguration, the device is adapted to work on larger blood vessels such as those encountered in vascular and transplant surgery.

The sensing concept is illustrated in FIG. 2E and the sensor apparatus structure is presented in FIGS. 2A-2C. As shown by FIGS. 2D and 2E, the arterial pulsation results in a change in vessel diameter, as shown by FIG. 2E, measured by the sensor apparatus 235 (e.g., a capacitive pulse sensor) mounted around the artery 237. The change in impedance, as shown by 241, results in a shift of the resonant frequency of the LCR circuit, as shown by 244. This shift is measured wirelessly through the skin 239 using an external reader coil 240, as shown by 245 of FIG. 2E. FIG. 2F illustrates an optical image 247 of a fabricated device with a close-up view of the sensor region of the device showing double capacitor structures with micro-structured PGS layer and scanning electron microscope image of the PGS layer showing pyramid structures. FIG. 2G illustrates example chemical structures of various polymers 202, 204, 206, 208, 210 which may be used to fabricate the sensor apparatus.

The sensor apparatus may include a fringe-field capacitive sensor (e.g., one or more capacitors) that is sensitive both in contact and non-contact modes, and a bi-layer coil structure that is used for RF data transmission. Due to the pulsatile nature of blood flow, the artery experiences a change in vessel diameter over time that is measured by the capacitive sensor. The change in impedance results in a shift of the resonant frequency of the LCR resonator circuit, monitored wirelessly through the skin via inductive coupling with an external reader coil in a battery-free approach. The equivalent electrical circuit is previously illustrated by FIG. 1C.

The sensor apparatus effectively includes a wireless circuit that consist of an LCR resonator, which the capacitor(s) connected in series with the inductive coils. The change in impedance due to artery expansion results in a shift of the resonant frequency ƒ₀ of the LCR resonator. This shift is monitored wirelessly through the skin by inductive coupling with an external reader coil, on the ports of which the scattering parameter, S₁₁ is measured. The proposed design allows for an easy fabrication process, and a resonant system with a high quality-factor, Q (corresponding to the low scattering parameter S₁₁ and narrow bandwidth), which is key to easily monitoring the impedance change of the sensor apparatus.

FIG. 2H illustrates a simplified view of a sensor apparatus, as is consistent with the sensor apparatus illustrated by FIG. 2A. The sensor apparatus 261 includes a sensor 269 and antenna 271 which are formed by respective portions of a patterned conductive layer 265. The sensor 269, antenna 271, and the patterned conductive layer 265 are laminated in an encapsulation 267. The antenna 271 includes the first and second inductive coils which are separated by the first elastomer material, as previously described. The sensor 269 includes first and second capacitors, as illustrated by the dielectric layers including pyramid microstructures 263. The patterned conductive layer 265 includes first and second wires that form the antenna 271 (e.g., the first and second inductive coils) and the sensor 269 (e.g., the first and second capacitors) which are coupled together, as previously described. The sensor apparatus 261 is laminated using the encapsulation 267 which includes a second elastomer material and a third elastomer material.

Sensor apparatuses in accordance with the present disclosure may have a variety of dimensions, and which be dependent on the specific application. For example, the sensor apparatus 261, as well as various embodiments of sensor apparatuses, may have a length 273 in a range of 0.5 millimeters (mm) to 10 centimeters (cm), a width 275 in a range of 0.1 mm to 5 cm, and a depth (or thickness) in a range of 10 nanometers (um) to 1 cm. For example, the length 273 may be not less than about 0.5 mm and not more than about 10 cm, a width 275 of not less than about 0.1 mm and not more than about 5 cm, and depth of not less than about 10 um and not more than about 1 cm. The antenna 271 (e.g., the inductive coils) may have width and length dimensions in a range of 0.1 mm to 5 cm. For example, the antenna 271 may have a width and length in a range of not less than about 0.1 mm and not more than about 5 cm. The width and length of the wires forming the patterned conductive layer 265, including portions of the antenna 271 and the sensor 269, may be in a range of 0.1 mm to 10 cm (e.g., width and lengths of not less than about 0.1 mm and not more than about 10 cm). The gap between the two wires forming the patterned conductive layer 265 may be in a um range, such as a range of 0.5 um to 100 um, and in other embodiments may be greater (e.g., a gap distance of not less than about 0.5 um and not more than about 100 um, although embodiments are not so limited). The length 273 may depend, for example, on the circumference of the targeted object (e.g., artery or other circular vessel) that the sensor apparatus 261 is to wrap around. For example, human arteries may be in a range of 1 to 3.6 mm. In some specific embodiments, the dielectric material overlapping the electrodes may include microstructures having dimensions in the um range, such as pyramids with widths and heights in a range of 0.1 um to 100 um and in specific embodiments a range of 0.5 um to 50 um. The microstructures may have a distance between adjacent microstructures in a range of 0.1 to 500-700 um, among other distances.

Although embodiments are not limited to the above dimension ranges, and sensor apparatuses may be formed in a variety of dimensions for different applications.

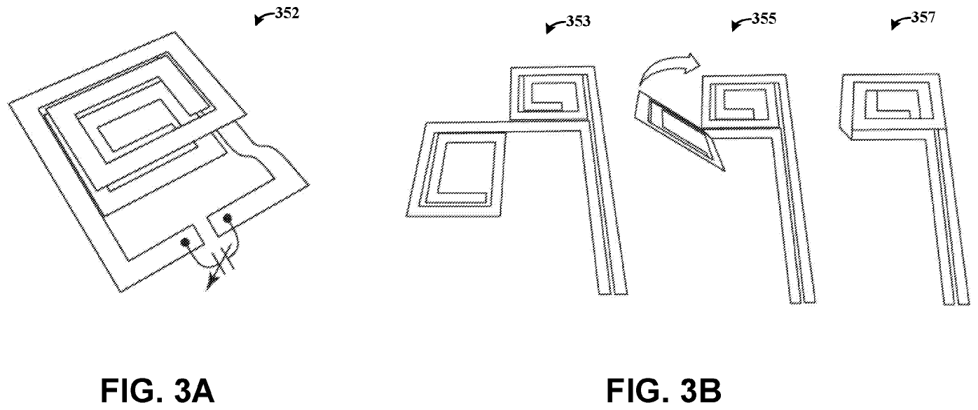

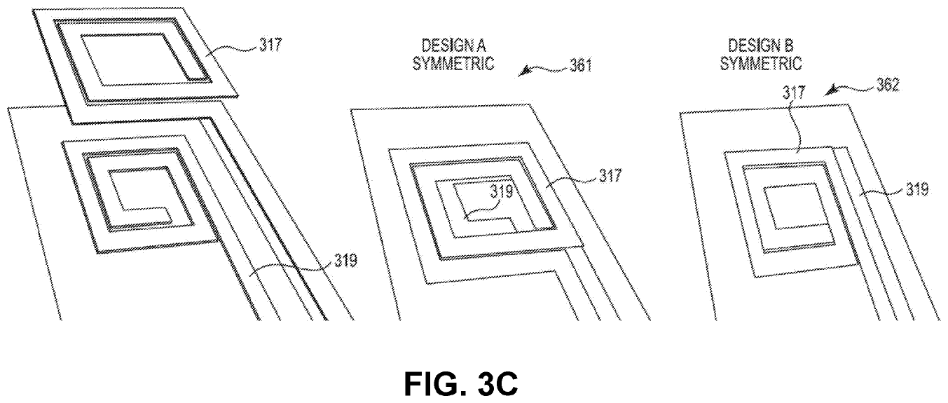

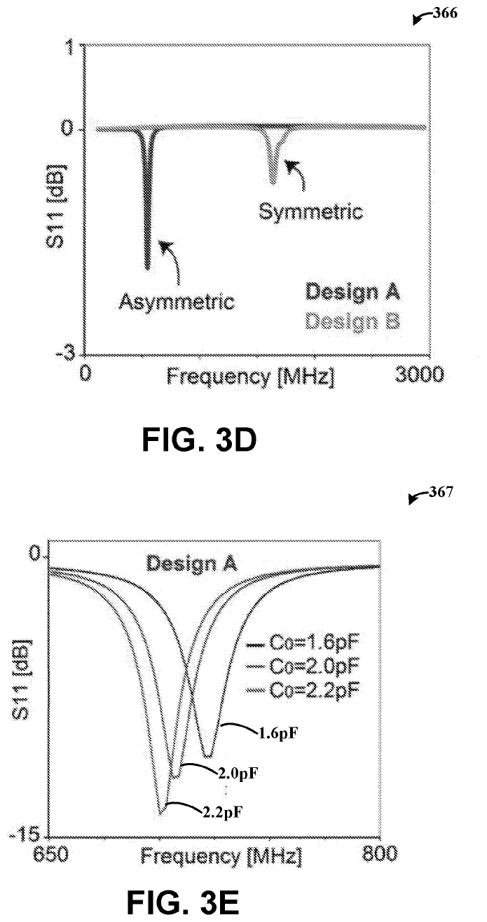

FIGS. 3A-3E show example inductive arrangements and forming of the same of a sensor apparatus, consistent with embodiments of the present disclosure. FIG. 3A shows an example of the LCR resonator circuit 352 that includes two inductive coils coupled to a capacitor.

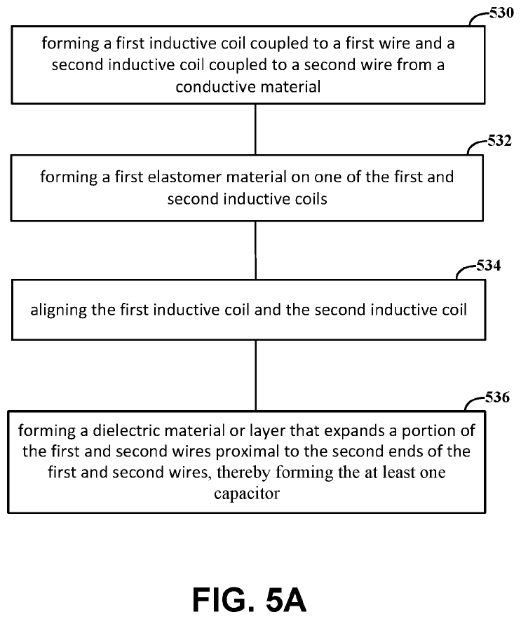

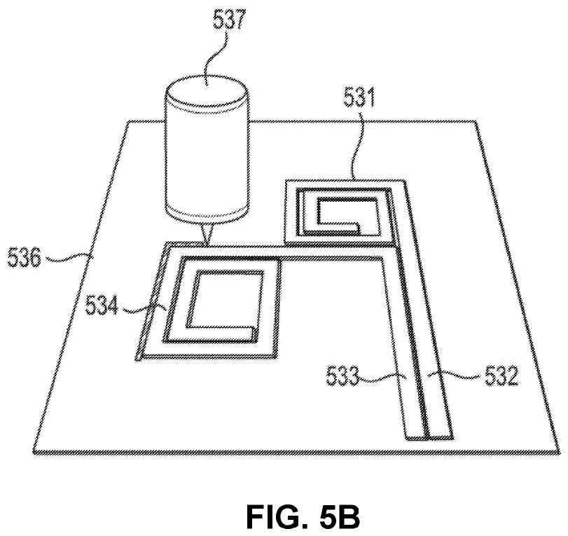

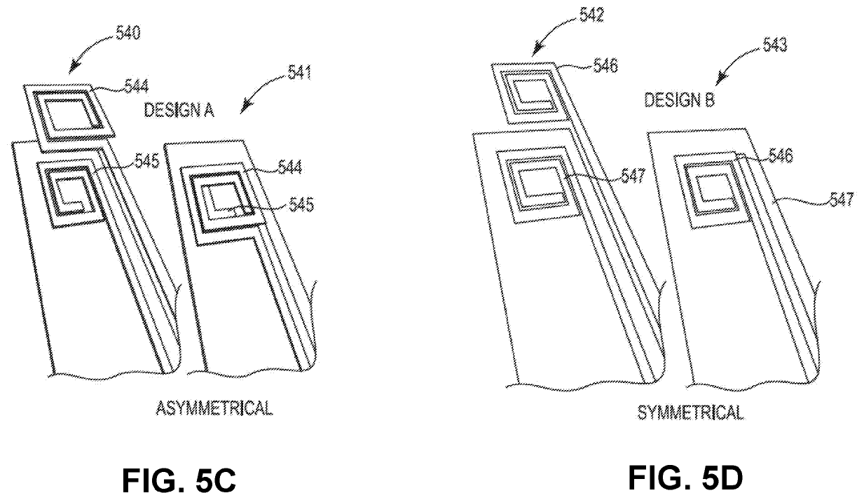

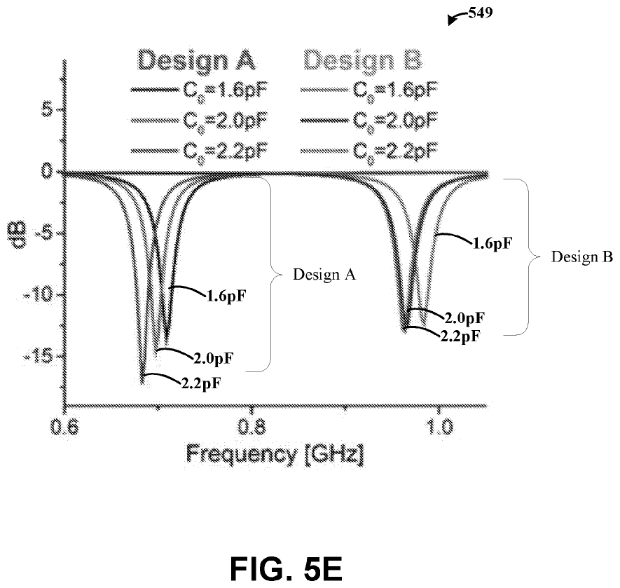

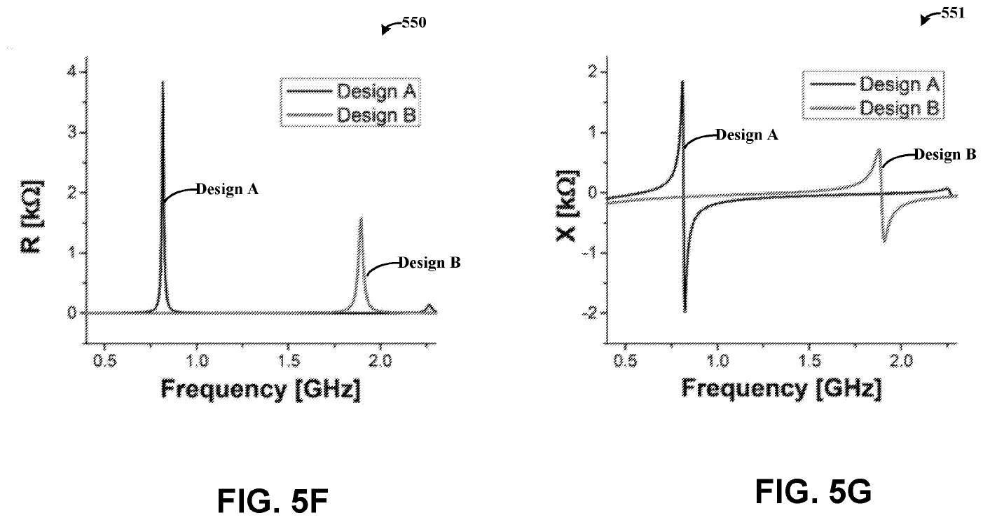

A typical flat LCR resonator consists of a capacitor connected in series with a planar coil that may require an additional delicate fabrication step to establish the electrical connection needed to close the LCR circuit. The illustrated LCR resonator circuit 352 does not include an interconnect and can be fabricated with a single fabrication step. Here, there is no need to establish any additional electrical connection, and the assembly is a simple lamination process of the two coils on top of one another, using a 50 μm-thick PLLA layer as an insulator. The metal lines are obtained via a one-step cutting process performed with a computer-controlled laser cutter, such as further illustrated by FIGS. 5A-5C, with the result illustrated by 353. The assembly and alignment of the coils is provided by the folding process as illustrated by 355 and 357 of FIG. 3B. The LCR resonator circuit 352, which is based on a bilayer coil structure, allows for a decoupling of the location of the pressure (or electromagnetic field) sensitive region from the location of the wireless link. This allows for freedom in the design of the implant to suit the specific biomedical application, in terms of geometry and dimensions.

FIG. 3C illustrates different designs of the inductive coils 317, 319. More specifically, FIG. 3C illustrates two designs 361, 362 for the coils 317, 319 (asymmetrical versus symmetrical) as evaluated with 3D electromagnetic field simulations. FIG. 3D is a graph 366 illustrating the evaluation of the two designs with 2D electromagnetic field simulations. The results, as illustrated by graph 366, show larger resonance shift for applied impedance change for design A 361 (asymmetrical) as compared to design B 362 (symmetrical). In addition, a larger and narrower S11 peak is obtained for design A 361 as compared to design B 362, corresponding to a larger Q factor as shown by FIG. 3D. The superiority of design A 361 is attributed to the quasi-closed large coil system, formed by the clockwise-spiraled top coil and the counterclockwise-spiraled bottom coil, the geometry of which is more favorable for the LCR resonator than that of design B 362. Moreover, a frequency shift in the order of tens of MHz may be obtained when varying the sensor's nominal capacitance C₀, and the resonant frequency ƒ₀ is lower in design A 361 than in design B 362. The resonant frequencies of both designs A 361 (asymmetric) and B 362 (symmetric) in combination with the primary side loop are depicted in FIG. 3D. The asymmetric design is more closely matched at the frequency of operation as depicted by the smaller return loss (S11).

FIG. 3E is a graph 367 illustrating electromagnetic simulation results for design A under different sensor capacitance loads for determination of resonant frequency of the device. Design A also provides for a greater shift in frequency for the same change in sensing capacitance, which provides more sensitivity. In addition, this design operates at a lower frequency, which is advantageous as RF signals suffer greater attenuation in the body at higher frequencies. Typically, the cost for operating at a lower frequency is a larger receiving loop. Designs A and B are the same size, and so no size penalty is incurred. This is highly desirable for wireless implantable systems with reduced losses in muscles, skin, and fat. Therefore, the most preferred design is that of Design A, the asymmetric configuration. Because RF signals are attenuated in tissue, this device will likely be limited to clinical applications in which the tag antenna could be placed close to the skin.

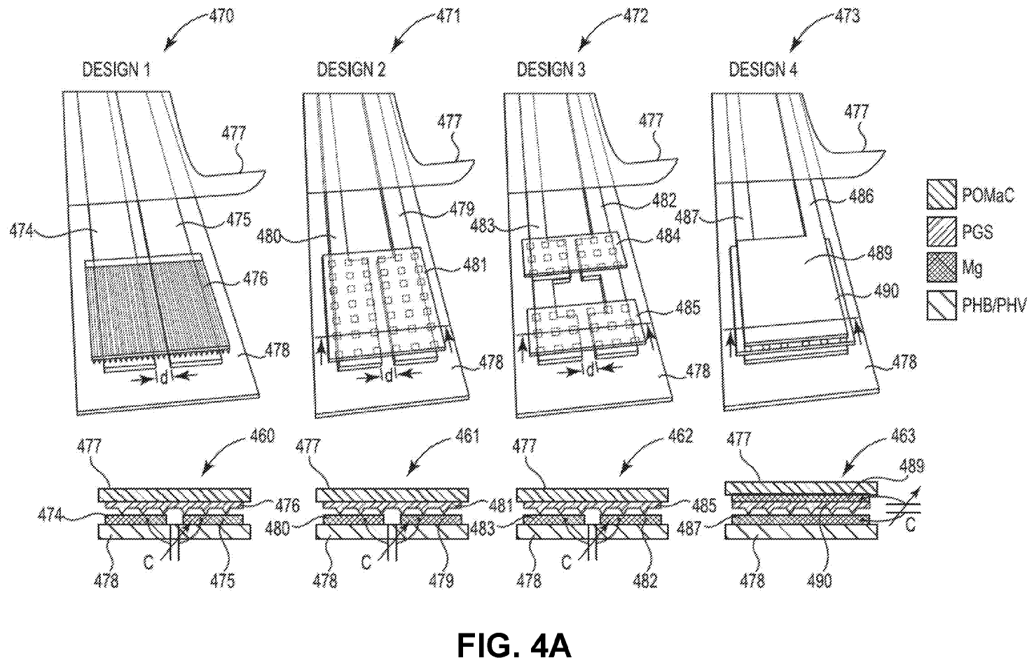

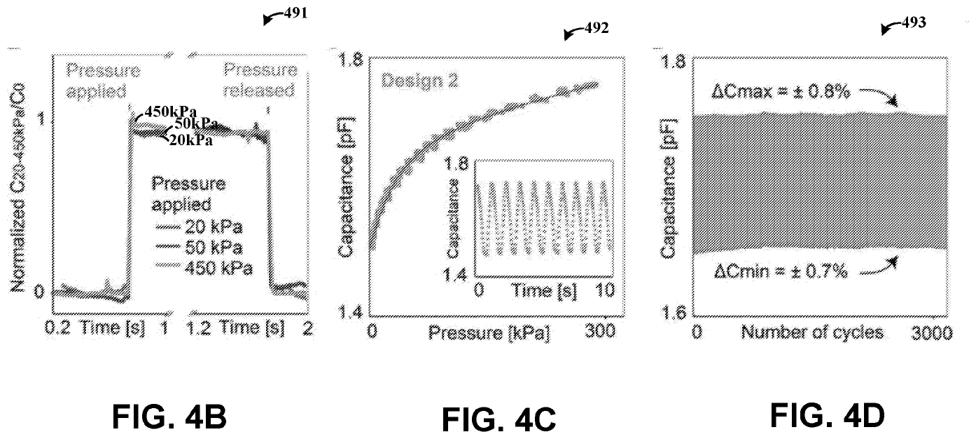

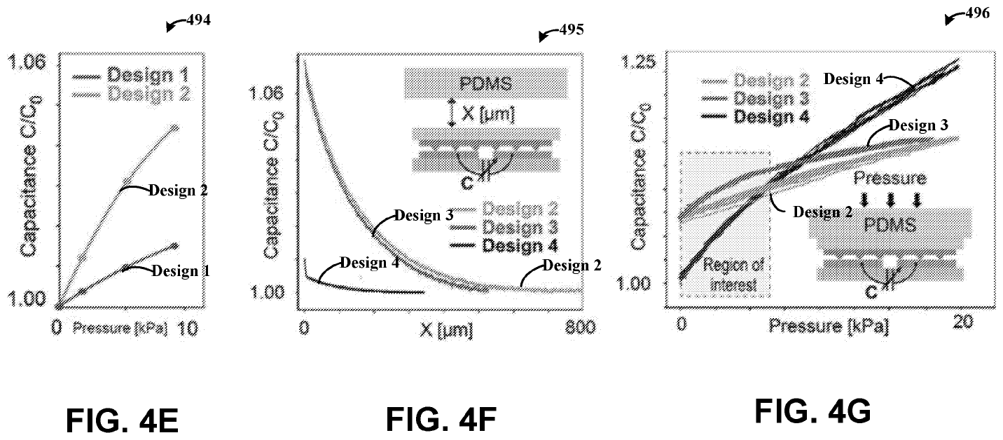

FIGS. 4A-4G show example capacitive arrangements of a sensor apparatus, consistent with embodiments of the present disclosure. One consideration in utilizing a cuff-type sensor apparatus around an artery is that the sensor cannot be secured too tightly so as not to block the blood flow within the vessel. The sensor apparatus in accordance with the present disclosure are designed such the apparatus can detect small pressures (below 5 kPa) in contact mode (the sensor is in contact with the artery), and can detect the expansion of the artery even though it is not in direct contact with the vessel in a non-contact mode.

Fringe-field capacitors are widely used for touch sensing applications because of their high sensitivity for detecting objects in close proximity. The shift in resonant frequency, Δƒ₀, due to capacitance change, AC, is calculated from the following equation:

$$Δ {f}_{0} = - \frac{Δ C}{4 π \sqrt{L {C}^{3}}}$$

The sensitivity of the capacitive sensor affects Δƒ₀ considerably and is maximized especially in the operation range of interest—specifically non-contact and low-pressure contact modes of operation.

Another consideration to designing the fringe field capacitive sensor is to maximize the sensitivity in contact mode. Finite element analysis (FEA) is used to characterize the electrostatic behavior of the system, and to investigate the effects of factors such as the dielectric material thickness, spatial density and height of pyramid structures, as well as the distance between the Mg electrodes as well as their orientation.

FIG. 4A illustrates four capacitor designs in accordance with various embodiments. In addition to the capacitor designs, the size of the microstructures of the dielectric layer may be adjusted, as further described herein. Each of the designs 1-4 is shown with a top down view 470, 471, 472, 473 and a side view 460, 461, 462, 463. The capacitor arrangement in the sensor apparatus may be used to measure pressure changes in contact (pressure sensing) and non-contact (fringe-field sensing) modes, such as for blood flow.

As shown by the top view 470 and side view 460, design 1 of the capacitor includes portions of the first and second wires 474, 475 (as coupled to the inductive coils not illustrated) and a dielectric material 476. The first and second wires 474, 475 and the capacitor are laminated between the second elastomer material 477 and third elastomer material 478 of POMaC and PHB/PHV, respectively. The dielectric material 476 includes PGS and the wires 474, 475 include Mg. The dielectric material 476 includes pyramid-shape microstructures with a base substrate width of 4 μm.

As shown by the top view 471 and side view 461, design 2 of the capacitor includes portions of the first and second wires 479, 480 (as coupled to the inductive coils not illustrated) and a dielectric material 481. The first and second wires 479, 480 and the capacitor are laminated between the second elastomer material 477 and third elastomer material 478 of POMaC and PHB/PHV, respectively. The dielectric material 481 include PGS and the wires 479, 480 include Mg. The dielectric material 481 includes pyramid-shape microstructures with a base width of 50 μm.

As shown by the top view 472 and side view 462, design 3 includes first and second capacitors, which each include portions of the first and second wires 482, 483 (as coupled to the inductive coils not illustrated) and first and second dielectric materials 484, 485. The first and second wires 482, 483 and the capacitors are laminated between the second elastomer material 477 and third elastomer material 478 of POMaC and PHB/PHV, respectively. The dielectric materials 484, 485 include PGS and the wires 482, 483 include Mg. The dielectric material 481 includes pyramid-shape microstructures with a base width of 50 μm.

As shown by the top view 473 and side view 463, design 3 includes a capacitor which include portions of the first and second wires 486, 487 (as coupled to the inductive coils not illustrated), a capacitive plate 489, and a dielectric material 490. The first and second wires 486, 487 and the capacitor are laminated between the second elastomer material 477 and third elastomer material 478 of POMaC and PHB/PHV, respectively. The dielectric material 490 include PGS and the wires 486, 487 include Mg. The dielectric material 490 includes pyramid-shape microstructures with a base width of 50 μm.

Each of the four designs are evaluated by applying pressure and releasing the pressure, as illustrated by FIG. 4B. FEA analysis, which combines both mechanical and electrostatic simulations, reveals that larger pyramid base widths of design 2 results in a sensor with at least twice the sensitivity of design 1, as shown by the graph 491 of FIG. 4B. This enhanced sensitivity is ascribed to the larger mechanical deformation of PGS in design 2 under the same applied pressure, both corresponding to a larger reduction in the air-gap and having an impact on fringe field. The response time in the millisecond range is illustrated by the graph 491. The response time and cycling durability satisfy the requirements for real-time monitoring of blood flow. FIG. 4C shows the pressure sensor response curves in design 2 from ten consecutive cycles with high reproducibility and negligible hysteresis. For example, the graph 492 of FIG. 4C illustrates, response characteristics of the pressure sensor, and more specifically, pressure response curve from ten consecutive cycles (applied pressure 0-300 kPa), displaying negligible hysteresis. FIG. 4D illustrates the sensor robustness under long cyclic pressure loadings, which are potentially encountered during operation. Experimental results show that the sensor can be reproducibly cycled thousands of times as shown by the graph 493. Cycling tests and stability of the pressure response (applied pressure 40 kPa to 230 kPa). FIG. 4E shows finite element simulation results of sensitivity analysis of design 1 and 2, as shown by the graph 494. For example, multi-physics simulation results with capacitance change as a function of applied pressure for design 1 and 2.

Further consideration is made for optimizing the sensor design to improve its flexibility to allow for wrapping around the circumference of a circular vessel as well as its sensitivity during non-contact regime. Two additional designs are compared to designs 1 and 2 (e.g., FIG. 4A). Design 1 or 2 has a single, large sensitive region while design 3 has two smaller sensitive regions, which compensate for bending effects required for securing the device around the artery and to prevent breaking of the PGS dielectric material. Design 4 is a standard double-plate-type capacitor. Increased thickness around the bent region makes it harder to wrap the sensor around the artery and increase in applied pressure for implantation caused delamination or breaking of the dielectric material for arteries with diameters less than 2 mm. Experiments are also conducted to investigate sensitivities for non-contact and contact mode operation. The results for non-contact mode operation are shown in the graph 495 of FIG. 4F, which shows measured sensor response to an object in close proximity (non-contact sensitivity) for design 2. The sensors response curves are measured while varying the distance between the sensor and an insulating polydimethylsiloxane (PDMS) block to mimic a potential object. The fringe field capacitive sensors (designs 1-3) have seven times higher sensitivities than the parallel plate capacitor for an object placed in close proximity.

The results for contact mode operation are provided by the graph 496 of FIG. 4G. After contact, the sensors response curves are measured while applying pressure. Design 4 shows a better sensitivity during contact mode. Given that contact between the sensor and the artery is not guaranteed and that relatively small pressures are expected during operation (below 5 kPa), the fringe field capacitive sensor of design 3 is identified as the design of choice for the following in vitro and in vivo investigations. Furthermore, using pyramid elastic structures in the sensing region and rigid PLLA material in between the coils at the wireless link, the sensor apparatus decouples the effect of pressure on the sensor and antenna. The design allows to convert an applied pressure on the sensor to an impedance change, while an applied pressure on wireless link does not cause the impedance change because of the rigid structure of the PLLA spacer.

FIGS. 5A-5G show example inductive arrangements of a sensor apparatus and resulting resonant frequency shifts, consistent with embodiments of the present disclosure.

More specifically, FIG. 5A shows an example fabrication method for a sensor apparatus. The sensor apparatus may allow for a LCR resonator to be formed with the additional connection to be formed to close the circuit. The additional connection is a metal interconnect, that can be made of the same material as the coil and capacitor, or another material, e.g., tin in case of soldering. This interconnect has to be electrically insulated from the conducting material it steps across, and the contact resistance at both ends of the interconnect is small and ohmic (not rectifying). The fabrication process in accordance with the present disclosure does not include this additional connection fabrication step.

The method includes forming a first inductive coil coupled to a first wire and a second inductive coil coupled to a second wire from a conductive material (e.g., laser cut Mg), at 530. As shown by FIG. 5B, forming the first and second inductive coils, the first wire, and the second wire may include laser cutting the first inductive coil coupled to the first wire and the second inductive coil coupled to the second wire from the conductive material, the first and second inductive coils being coupled together. The method further includes forming a first elastomer material on one of the first and second inductive coils, at 532, and aligning the first conductive coil and the second conductive coil such that the first elastomer material is there between and the first and second wires extend from the first and second inductive coils at first ends of the first and second wires with a distance between at second ends of the first and second wires, at 534. In various specific embodiments, aligning the first and second inductive coils includes folding the second inductive coils to align with the first inductive coil.

The method further includes, at 536, forming a dielectric material or layer that expands a portion of the first and second wires proximal to the second ends of the first and second wires, wherein the portion of the first and second wires form at least one capacitor of the sensor apparatus configured to exhibit a change in impedance in response to a pressure-manifestation change associated with an object, and the change in impedance is to cause a change in the resonant frequency of the first and second inductive coils. The at least one capacitor, while the sensor apparatus is in use, is configured to at least partially wrap a circumference of the object. The first and second inductive coils form an antenna and the at least one capacitor forms a sensing region of the sensor apparatus. In specific embodiments, the sensing apparatus includes two capacitors which are formed by a first dielectric material and a second dielectric material which span different portions of the first and second wires, as previously described.

The method may further include laminating the sensor apparatus. For example, the sensor apparatus is laminated with a second elastomer material proximal to one of the first and second inductive coils and the dielectric material (e.g., the soft POMaC layer) and a third elastomer material (e.g., the relatively stiffer PHV/PHB layer) proximal to the second of the first and second inductive coils. Each of the various elastomer materials may be biodegradable, as previously described.

FIG. 5B illustrates an example of laser cutting the LCR resonator circuit. As shown, the conducting lines for electrical interconnects, capacitors and inductors are defined using a one-step fabrication process, with a computer-controlled laser cutter 537 (Epilog Fusion M2 Fiber Laser operated at 40 W) which cuts the inductive coils 531, 534 and the wires 532, 533 from the Mg foil 536.

FIGS. 5C-5D illustrate two designs for the inductive coils. FIG. 5C illustrates a first design (e.g., design A) in which the top and bottom coils 544, 545 are asymmetrical, with the first view 540 showing the coils 544, 545 separate and the second view 541 showing the coils 544, 545 aligned. The top and bottom coils 544, 545 are asymmetrical in that the top coil 544 turns clockwise while the bottom coil 545 turns counterclockwise, forming a quasi-closed large coil when superimposed on each other. In many embodiments, the design with the lower resonant frequency and a larger resonant frequency shift may be used, which may include the first design (which may be referred to as design A).

FIG. 5D illustrates a second design (e.g., design B) in which the top and bottom coils 546, 547 are symmetrical, with the first view 542 showing the coils 546, 547 separate and the second view 543 showing the coils 546, 547 aligned. The top and bottom coils 546, 547 are symmetrical in that the top coil 546 and bottom coil 547 turn the same way, forming a quasi-closed large coil when superimposed on each other.

FIG. 5E is a graph 549 showing simulated S11 return loss for whole system when tagged with sensor is coupled to reader coil for both the first and second designs. A reader port is referenced to 50 Ohms. Given similar sensor capacitance changes shift for the first design is more linear (e.g., asymmetric). FIG. 5F is a graph 550 illustrating electrical simulation results of the first and second designs showing resistance and FIG. 5G is a graph 551 showing reactance of the first and second designs at the sensing capacitance port with no coupled reader coil.

Relating to the above applications, according to certain specific embodiments, a biodegradable sensor apparatus is configured and arranged to monitor locally and in real-time the blood flow in an artery, allowing for wireless monitoring of vessel anastomosis after surgery. There is no need of additional surgery to remove the sensor after its period of use, and it is completely made of biodegradable and biocompatible materials. Experimental embodiments in this regard have been demonstrated so as to provide sensor operation with fast response time (in the millisecond range), high cycling durability (over thousands of cycles), negligible hysteresis, high robustness (from low to high applied pressures as potentially encountered during surgical operation), that satisfy the requirements for real-time monitoring of blood flow. Such a sensor apparatus may be biodegradable, flexible, wireless and battery-free, which is mounted on an artery and used for various surgical operations. The sensor apparatus may be used to measure the pulse rate (as e.g., with Doppler-based approaches) and/or the pulse shape (e.g., width, length, and waveform of the pulse), which is additional information useful for surgeons to improve the quality of post-operative monitoring. The sensor is based on fringe field capacitor technology and senses arterial blood flow both in contact and non-contact modes, allowing for easier mounting and reducing the risk of vessel trauma. The sensor apparatus can measure the blood flow both in contact (pressure sensing) and non-contact (fringe-field sensing) modes.

### Experimental/More-Detailed Embodiments

Certain specific embodiments of the instant disclosure are directed to the area of vessel anastomosis as part of important procedures in cardiovascular, vascular, and transplantation surgeries. For such surgeries, there are a multitude of patients who undergo coronary artery bypass grafting annually, and with an expanding number due to population aging. Other common diseases requiring revascularization include limb ischemia (CLI) and traumatic vascular repair surgeries. Additionally, there is an expanding role for microvascular free tissue transfer to reconstruct patients after cancer or trauma. After each of these operations, ensuring blood flow through the newly created anastomosis is important. However, post-surgical monitoring of vessel anastomosis is inconsistently done. Often, failure is not noted until the opportunity to save the graft has passed, making repeat intervention required. After reconstructive surgery involving microsurgical anastomosis, failure occurs via formation of a hematoma or thrombosis within the artery or vein. It has been shown that surgical salvage is closely correlated with the intensity of monitoring and time from detection to return to the operating room. The proposed biodegradable sensor can be used to wirelessly monitor the arterial blood flow after microvascular reconstruction surgery continuously, eliminating a lag time between loss of blood flow and detection.

According to certain specific embodiments, advantages regarding type of signal recorded are provided. These include, for example, methodology to record the pulse rate (e.g., with Doppler-based approaches) and also the pulse shape (width, length, and waveform of the pulse), which is additional information useful for surgeons to improve the quality of post-operative monitoring.

According to certain other/related specific embodiments, there are advantages regarding the sensing mode used for such monitoring and data collection. The sensor is based on fringe field capacitor technology and senses arterial blood flow both in contact and non-contact modes, allowing for easier mounting and reducing the risk of vessel trauma. The device can measure the blood flow both in contact (pressure sensing) and non-contact (fringe-field sensing) modes. In this context, fringe-field sensing refers to, includes, and/or is exemplified by circuitry, materials and field-based changes (e.g., capacitance versus distance and otherwise) as discussed in connection with specific example embodiments in the second or supplemental Appendix which forms part of this patent application; see, e.g., disclosure in connection with FIGS. 3A-3G of the second or supplemental Appendix of the underlying Provisional Application.

According to certain other/related specific experimental embodiments, a double-inductor/bilayer coil structure is used with a pressure sensitive elastomer laminated between two coils. Example applications include use of this construction to monitor intracranial pressure changes wirelessly. The bilayer coil structure allows for a decoupling of the location of the pressure sensitive region from the location of the wireless link. This decoupling feature gives additional freedom in the design of the implant to suit the specific biomedical application, in terms of geometry and dimensions (smaller sensor at the location of artery and better wireless link with more flexibility in the positioning of the wireless double-coils).

According to certain other/related specific embodiments, such features and methodology allows for an easy fabrication process. The sensor assembly is a simple bench-top process involving lamination and packaging with a UV-cured biodegradable sealant. Such a device can be fabricated by laminating and interconnecting operations/steps as exemplified in the Appendix, for yielding impressive sensor sensitivity and response-time operation, for example, using a construction having pyramids molded from a silicon mold. In one such highlighted embodiment, the packaging consists of a soft elastomeric POMaC layer that is in contact with the artery, while the stiffer PHB/PHV layer is in contact with the surrounding muscles, producing a device that is more sensitive to artery expansion than respiratory motion. The PLLA spacer is configured and arranged to prevent electrical short between the coils.

As an example, in accordance with various embodiments, the sensor fabrication is a bench-top process involving lamination and packaging with a UV-cured biodegradable sealant. The device is fabricated by laminating the 50 μm-thick Mg electrical interconnects together with the 40 μm-thick PGS dielectric layer used for the pressure sensitive regions, the 10 μm-thick POMaC and 10 μm-thick PHB/PHV packaging layers, and the 50 μm-thick PLLA spacer used for the bilayer coils. The PGS dielectric layer is micro-structured to allow for improved sensor sensitivity and response-time, with pyramids molded from a silicon mold. The packaging consists of a soft elastomeric POMaC layer that is in contact with the artery, while the stiffer PHB/PHV layer is in contact with the surrounding muscles, producing a device that is more sensitive to artery expansion than respiratory motion. The PLLA spacer prevents electrical shorts between the coils. The device flexibility is further improved by having two narrow variable capacitors instead of a single larger one, allowing for easy sensor wrapping around arteries with a diameter of less than 1 mm.

Other related advantages ensuing from such fabrication process and structure are as follows. There is no need of an additional fabrication step with electrical connection to close the LCR resonant circuit. A typical flat LCR resonator consisting of a capacitor connected in series with a planar coil requires an additional delicate fabrication step to establish the electrical connection needed to close the LCR circuit, according to certain specific embodiments, a LCR resonator is used without need to establish any additional electrical connection, and the assembly is a simple lamination process of the two coils on top of one another, using a PLLA layer as an insulator. The metal lines are obtained via a simple one-step cutting process performed with a computer-controlled laser cutter. The easy assembly and alignment of the coils is facilitated by the folding process as described in the abstract of the Appendix of the underlying Provisional Application.

Additional advantages concern sensing performance and wireless circuit optimization. For one such related embodiment, an optimization of the wireless circuit design is performed where the design allows for a resonant system with high quality factor Q, which is discovered as being important to monitor the capacitance change(s) of the sensor with relative ease. Such related embodiments more specifically include two example designs for the coils (asymmetrical versus symmetrical, design A and B, respectively), and each is evaluated with 3D electromagnetic field simulations. The results show larger surface current densities at resonance for one such design (design A) as compared to the other design (design B). In addition, a larger and narrower S11 peak is obtained for design A as compared to design B, corresponding to a larger Q factor. The superiority of design A (asymmetrical) is attributed to the quasi-closed large coil system, formed by the clockwise-spiraled top coil and the counterclockwise-spiraled bottom coil, the geometry of which is more favorable for the RLC resonator than that of design B. Moreover, a frequency shift in the order of several tens of MHz is obtained when varying certain factors such as the sensor current and the nominal capacitance (C₀), and the resonant frequency f0 is lower in design A than in design B. This is an asset because the lower operational frequency results in a wireless system with reduced losses in muscles, skin, and fat.

For yet another effort concerning related embodiments, an optimization of the fringe field capacitive sensor design is performed, with the objective to maximize the sensitivity of the sensor and allow for easy sensor wrapping around arteries with a diameter of less than 1 mm. In this regard, the flexibility of the device and materials can play a role. The first step consists of designing the fringe field capacitive sensor to maximize the sensitivity in contact mode. Finite element analysis (FEA) is used to characterize the electrostatic behavior of the system, and to investigate the effects of factors such as the dielectric layer thickness, spatial density and height of pyramid structures, as well as the distance between the Mg electrodes. The second step consists of further optimizing the sensor design to improve its flexibility to allow for wrapping around the circular vessel. A new design with two smaller sensitive regions is found. This allows to compensate for bending effects required for securing the device around the artery and to prevent breaking of the PGS dielectric layer.

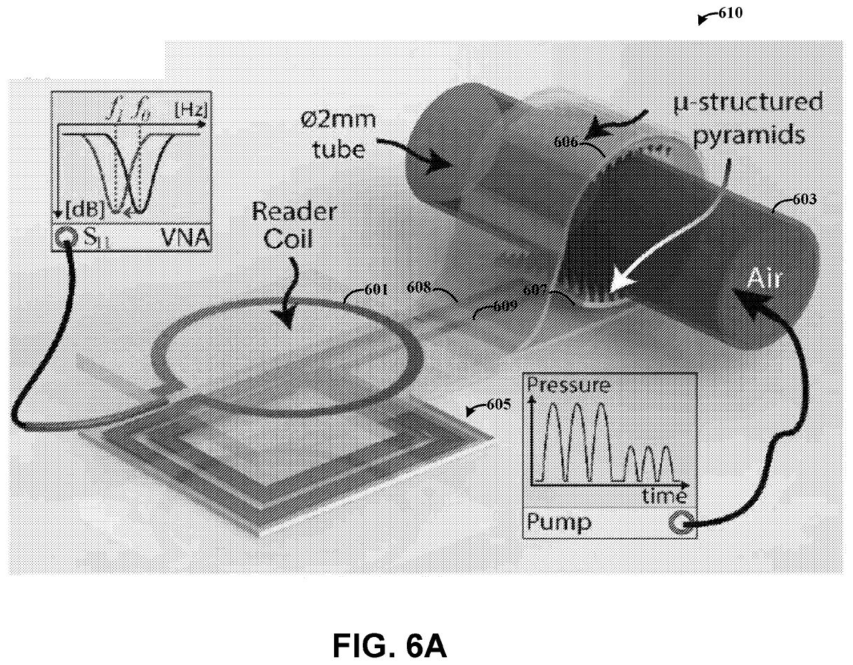

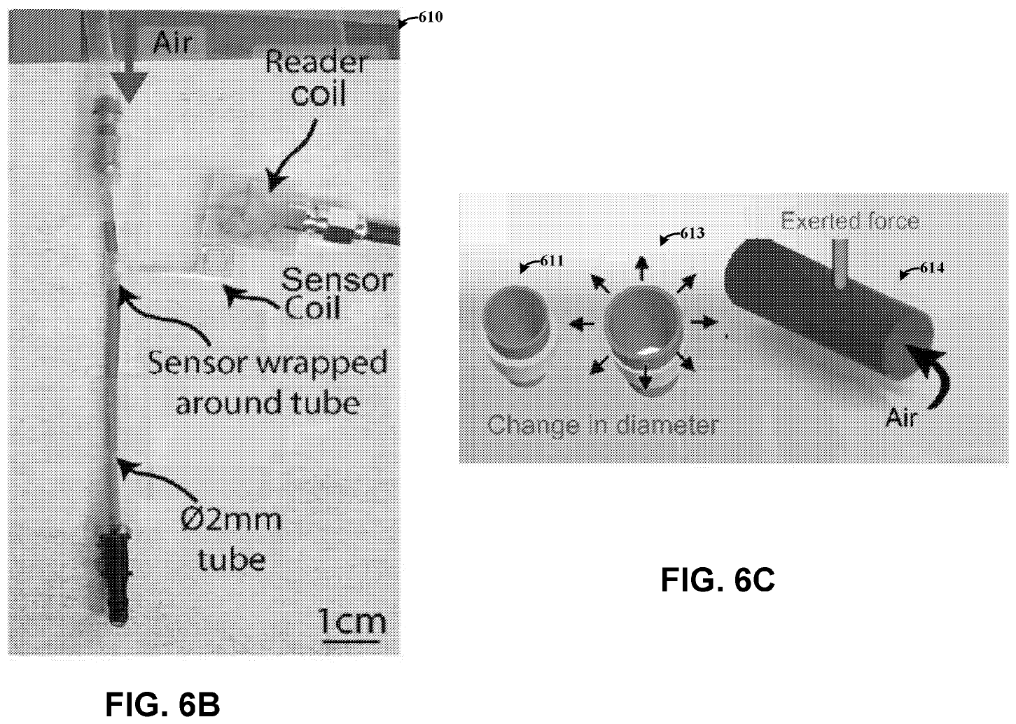

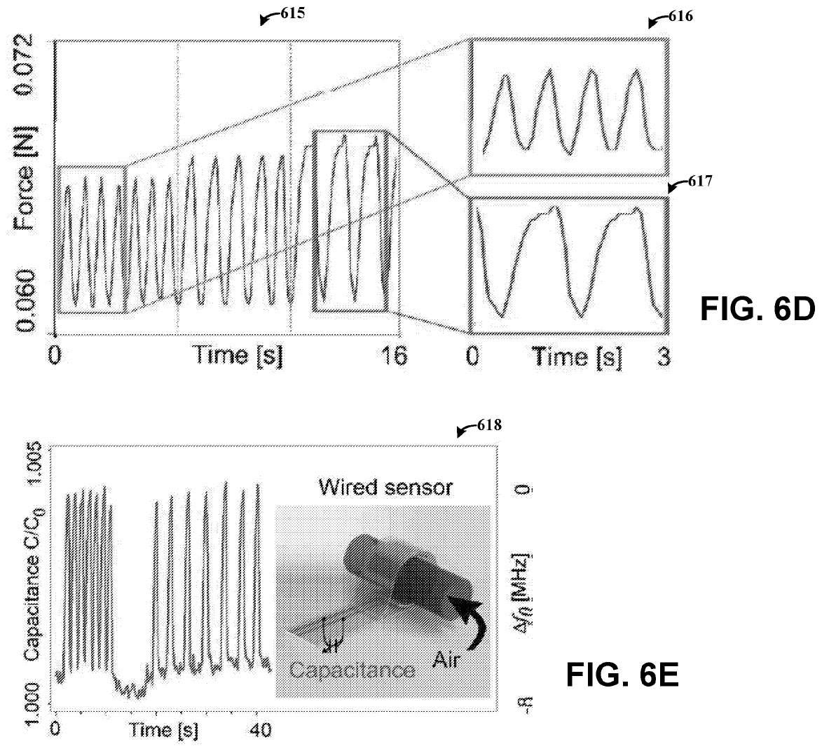

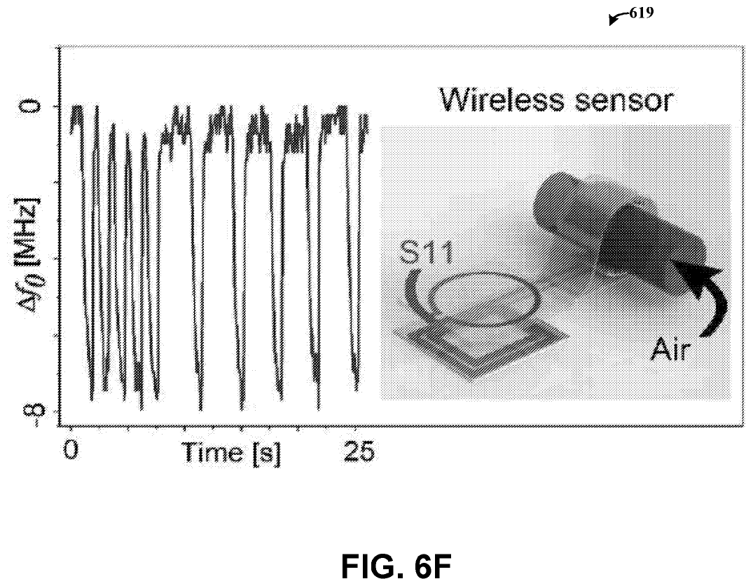

FIGS. 6A-6F show an example of a sensor apparatus under different forces, consistent with embodiments of the present disclosure. The sensors are first characterized in vitro on a model that mimics the pulsatile behavior and typical expansion of the facial artery. In the wireless sensor configuration, shown by the sensor apparatus 610 of FIGS. 6A-6B, the fringe field capacitive sensor, e.g., the first and second capacitors 606, 607 coupled to the inductive coils 605 via the wires 608, 609, is wrapped around a 2 mm-diameter polyolefin tube 603, which is closed at one end and connected to an air pump at the other. The reader coil 601, connected to a vector network analyzer (VNA), allows for the wireless measurement of the resonant frequency of the sensor in real-time (S11 scattering parameter). More specifically, FIG. 6A illustrates a schematic of the sensor apparatus 610 and the experimental set up, and FIG. 6B illustrates an optical image of the sensor apparatus 610 and the experimental set up. The test setup mimics the pulsatile behavior and typical expansion of a facial artery. The biodegradable wireless fringe field capacitive sensor is wrapped around a 2 mm-diameter polyolefin tube which is closed at one end and connected to an air pump at the other. The reader coil, connected to a vector network analyzer, allows for the wireless measurement of the resonant frequency of the sensor in real-time.

The custom-made artificial artery model is designed to mimic the properties of the facial artery, in terms of diameter (typical facial artery diameter ranges from 1.7 to 3.6 mm in adult), variation in vessel diameter with blood flow (below 4%), and pulse frequency (within normal resting heart rate for adults). The artificial artery is selected due to its small diameter as well as natural application of a wireless biodegradable sensor on this vessel after facial reconstruction. The setup is characterized by measuring the change in diameter and the expansion force exerted by the artificial artery model with various pulsed air frequencies and amplitudes (e.g., FIGS. 6C-6D), showing performances well within the characteristics of a real facial artery. More specifically, FIG. 6C illustrates characterization of the artery model including a change in diameter of the artery illustrated by 611, 613 and the exerted force illustrated by 614. FIG. 6D is graph 615 showing measured expansion force, calculated from the calibration curve for the sensor, exerted by the artificial artery tube with various pulsed air frequencies and amplitudes. The left side is illustrated by the insert 616 and includes setting 1, 75 pulses/min, middle setting 2, 57 pulses/min, and the right side is illustrated by the insert 617 and includes setting 3, 43 pulses/min. The maximum variation of tube diameter (setting 3) is measured to be 34 μm corresponding to an artificial artery expansion of −2%, well within the characteristics of an example facial artery.

As illustrated in FIGS. 6E and 6F for a wired and wireless sensor, respectively, when air is pumped through the tube, it expands and this expansion causes compression of the sensitive micro-structured PGS dielectric layer resulting in an increase in effective dielectric constant of the fringe-field capacitive sensor. This change in the dielectric layer structure causes an increase in capacitance, which in turn causes a decrease in the resonant frequency of the wireless device. This is reflected in a shift in resonance of the overall system. For example, FIG. 6E is a graph 618 illustrating capacitance measured for a wired sensor wrapped around the artificial artery. The wired design is the same as the wireless sensor, except that the two coils are replaced by conductive lines connected to an LCR-meter, allowing for the measurement of the capacitance in real-time. FIG. 6F is graph 619 illustrated the shift of the resonant frequency measured for a wireless sensor wrapped around the artificial artery, with various pressure patterns applied.

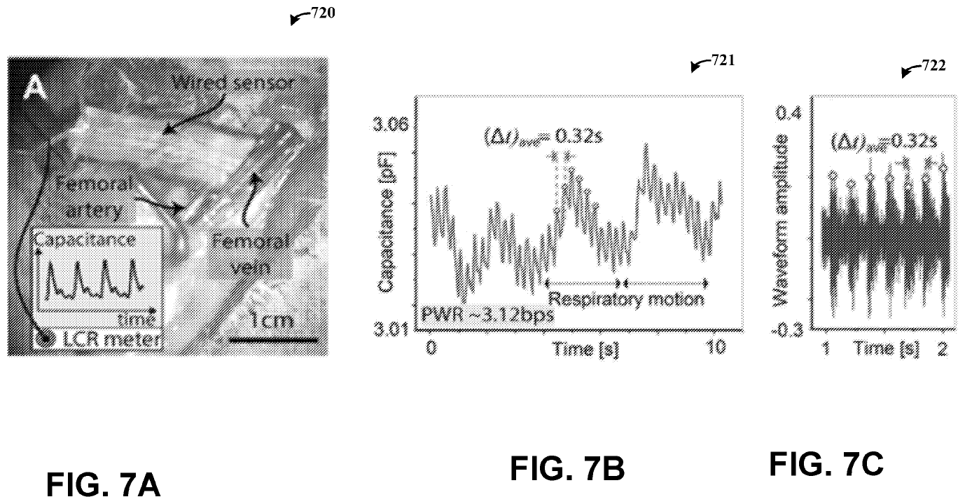

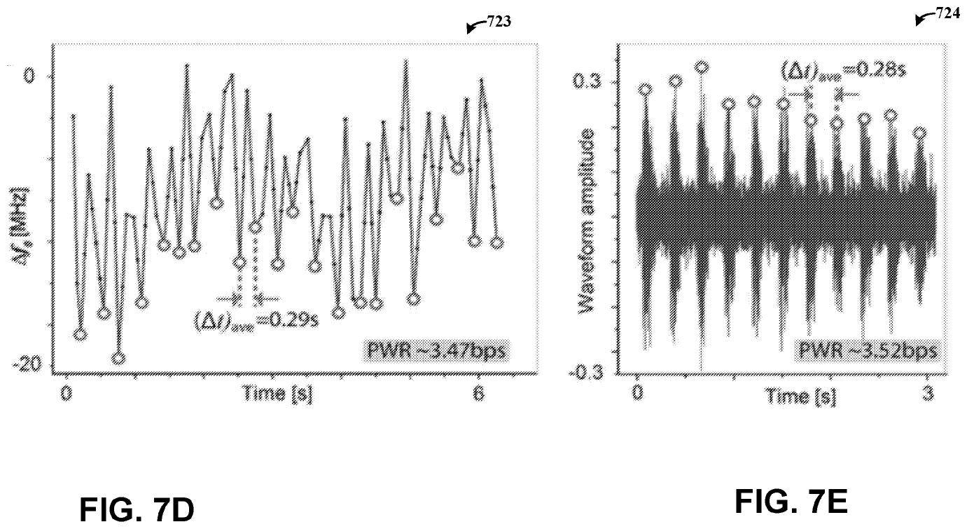

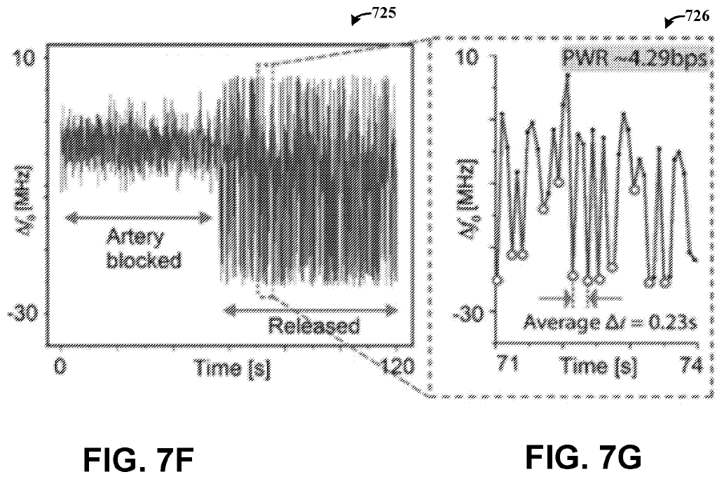

FIGS. 7A-7G show another example sensor apparatus and responses to pressure applied, consistent with embodiments of the present disclosure. Various experimental embodiments are directed to in vivo blood flow monitoring using example sensor apparatuses. The in vivo sensor operation in wired and wireless configurations is illustrated in FIG. 7A. These configurations are used to verify the proper operation of the sensing capacitor for blood flow monitoring and validate the wireless link, respectively.

A typical facial artery is approximately 1-2 mm in diameter depending on the age and size of the patient. The rat femoral artery is slightly smaller closely mimicking a pediatric facial artery. The pulsatile behavior is similar to that seen in the facial artery of humans. A smaller vessel is selected as detecting small changes in diameter would be more difficult in a small vessel. Additionally, background alterations in vessel diameter due to respirations would also be important to distinguish in a small vessel. Moreover, the femoral artery is located between hip and knee joints. This is a very challenging location for sensor durability test, because joint movement will cause additional physical stress not only to the sensor but also to the artery wrapped around by the sensor. By making the model more challenging, the sensor apparatus may be ensured to work on larger vessels and when the sensor is implanted around joints.

FIGS. 7A-7C show in vivo arterial pulse monitoring with a wired sensor implanted in a rat. A sensor with dimensions 5 mm×20 mm is wrapped around a Sprague Dawley rat femoral artery and fixed by sutures as showed by the optical image 720 of FIG. 7A of the implantation site with the wired sensor wrapped around the femoral artery and fixed with sutures. The magnesium wires of the sensor are connected to an LCR-meter for capacitance measurement. After the implantation, the sensor is connected to an LCR-meter to monitor the changes in sensor capacitance, and a pulse rate of 3.12 beats per second (bps) is recorded, as shown by the graph 721 of FIG. 7B. That is, the graph 721 of FIG. 7B illustrates measured capacitance of implanted wired sensor during in vivo tests. Measurements of the pulse rate are performed simultaneously with a standard external Doppler ultrasound, shown by the graph 722 of FIG. 7C, and confirm the results obtained with the wired sensor. Respiratory motion is also visible in FIG. 7B, but can be clearly distinguished from the arterial pulse-wave. The proposed packaging with soft POMaC in contact with the artery and a stiffer PHB-PHV thin film exterior to the sensor results in an improved sensitivity towards blood flow monitoring. Graph 722 of FIG. 7C illustrates measured sound waveform of the external Doppler ultrasound during the capacitive sensor measurement. A microphone is used to record sounds generated from the Doppler ultrasound and analyzed on computer for pulse rate detection.

FIGS. 7D and 7E illustrate the ability of the device to monitor the artery pulse-wave wirelessly (n=3). A sensor with dimensions 5 mm×20 mm (total length) with a 10 mm×10 mm coil structure is implanted in a rat similar to the wired configuration. An external reader coil is inductively coupled with the sensor coil structure implanted below the skin (e.g., FIGS. 5E-5F of the underlying provisional application), and the S11-parameter is recorded with a network analyzer, as shown by FIG. 7D. An external Doppler ultrasound is also used to monitor the pulse for comparison, as shown by FIG. 7E. FIG. 7D shows a graph 723 illustrating the measured shift in resonant frequency (e.g., measured resonance frequency shift versus time plot; the pulse-wave rate is calculated to be 3.47 bps). The minimum peaks (circles) correspond to the artery expansion with pulsation and increasing sensor capacitance. The pulse rate is calculated to be 3.47 bps. The simultaneously recorded and analyzed sound waveform, as shown by the graph 724 of FIG. 7E, indicates a pulse rate of 3.52 bps, which is very close to the value obtained via wireless sensor (e.g., measured sound waveform of the external Doppler ultrasound through a microphone; the pulse-wave rate is calculated to be 3.52 bps). The slight difference can be explained by the relatively slower sampling rate of the network analyzer relative to external Doppler ultrasound and microphone, which can be improved with a customized network analyzer system.

To further demonstrate the performance of the wireless blood flow sensor, an occlusion test is performed (as illustrated by FIGS. 5I-K of the underlying provisional application) whereby the femoral artery is blocked for 1 minute and then released. The objective is to investigate the response of the wireless device to change in the artery flow. In certain medical conditions, early clot forming within the vessel slows down the flow of blood through the artery and decrease expansion of the artery diameter. By applying tension to the artery, such conditions are mimicked and performance of the sensor is observed. The sensor is implanted as described earlier. Two nylon sutures are placed around the femoral artery at each side of the sensor (in red in FIG. 5I of the underlying provisional), and tension is applied to obstruct arterial flow. An external reader antenna is used to monitor the S11 parameter with a VNA. FIG. 7F is a graph 725 showing 2-minute long measured resonant frequency shifts of the wireless device. The graph 725 shows after release of the sutures occluding the artery amplitude of the artery's resonant frequency shifts increased considerably. The graph 725 is thereby a two-minute long plot showing the measured resonant frequency shifts as a function of time for the wireless sensor using the VNA. The first minute corresponds to when tension is applied through sutures, after which the femoral artery is released. FIG. 7G is a graph 726 showing a close-up view of the measurements for a shorter period after the release of the sutures. The pulse rate is calculated by averaging the time difference between peaks. The resulting pulse rate of 4.29 bps matches closely with the recorded ultrasound measurements of 4.33 bps, demonstrating the proper operation of the wireless device. The small increase in pulse-rate can be explained by the obstruction of the artery for a short time, verified in both sensor and Doppler measurements.

To demonstrate sensor function over time, the sensor is implanted for one week and then tested. After one week of implantation, wireless monitoring of the artery expansion yielded similar results, with measured pulse rates closely matching that obtained by external Doppler ultrasound. The only difference is the lower quality factor of the signal, likely due to presence of moisture. After 12 weeks of implantation, the rat is able to move without any apparent limb impairment. The sensor is harvested after 12 weeks, and partial degradation of the sensor is observed. All components including the POMaC sealing layer, PGS micro-structured pyramids, Mg wires and PLLA thin film are degraded, and only PHB/PHV is left, in accordance with the reported relative different degradation rates of these materials. The duration for degradation can be further tuned by varying the thicknesses of the layers, e.g., if slower degradation is required. The harvested sensor sample, together with the surrounding artery and tissues, are investigated for any foreign body reaction. Histological evaluations evidenced the patency of the artery and no severe inflammation around the sensor implantation site.

Experimental embodiments are directed to a novel pressure sensor entirely comprised of biodegradable materials and based on fringe field capacitive sensing. The device can be operated wirelessly via inductive coupling. In addition to investigations of the key engineering aspects and design optimization for the wireless circuit and fringe field capacitive sensor, the operation of wired and wireless devices is demonstrated using a mock setup as well as in vivo in a rat model. The device allows for short-term monitoring of vessel patency. Being biodegradable, it does not require a second procedure for implant removal. This sensor has multiple applications to both small and large vessels after surgical procedures requiring vessel anastomosis including cardiac, vascular, transplant and reconstructive operations.

As described above, various polymers may be used to form the sensor apparatus. The following is an example synthesis of POMaC pre-polymer. POMaC is synthesized as described in Tran, R. T., et al., “Synthesis and characterization of a biodegradable elastomer featuring a dual crosslinking mechanism,” Soft Matter 6 2449-2461 (2010), which is incorporated herein in its entirety for its teaching. Briefly, maleic anhydride (Fluka, CAS 108-31-6), citric acid (Sigma Aldrich, CAS 77-92-9) and 1,8-octanediol (Sigma-Aldrich, CAS 629-41-4) are mixed in a 3 necked round-bottom flask with a molar feed ratio of 3:2:5, respectively. The flask content is heated at an initial temperature of 160° C. and stirred under nitrogen atmosphere. After the mixture is melted, the temperature is set to 140° C. and continuously stirred under nitrogen for 3 hours. To remove unreacted monomers and oligomers, the pre-polymer is dissolved in tetrahydrofuran (THF, about 5 g in 20 ml), and purified by drop-wise precipitation into 2 liters of deionized water.

The following is an example fabrication of a POMaC packaging layer. Photocrosslinked POMaC networks (PPOMaC) are formed by crosslinking through free radical polymerization. The photoinitiator 2-Hydroxy-4′-(2-hydroxyethoxy)-2-methylpropiophenone (Sigma Aldrich, CAS 106797-53-9) is dissolved in 1,4 dioxane (5 ml) and mixed with pre-POMaC (5 g) using a speed mixer at 3000 rpm overnight. The solution is then spin-coated on a Si wafer, first at 500 rpm for 45 seconds and then at 1500 rpm for 1 minute. The sample is then exposed to a 365 nm ultraviolet light lamp (10 mW/cm² during 4 minutes) as described in Hoa, J. S. et al., “Wireless power transfer to deep-tissue microimplants,” PNAS 111(22) 7974-7979 (2014), which is incorporated herein in its entirety for its teaching.

The following is an example sensor assembly. The PGS micro-structured layer with pyramids is fabricated as described in Boutry, C. M., et al., “A Sensitive and Biodegradable Pressure Sensor Array for Cardiovascular Monitoring,” Adv. Mater. 27 6954-6961 (2015), which is incorporated herein in its entirety for its teaching. It is laminated on top of the POMaC polymerized thin-film, together with the Mg-foil structure cut as described further herein. Mg foils (50 μm-thick) are laser cut (Epilog Fusion M2). POMaC layer is spin-coated at 500 rpm for 45 seconds and then at 1500 rpm for 1 minute to yield ˜35 μm thickness. The sample is exposed to a 365 nm ultraviolet light lamp (10 mW/cm² during 4 minutes) as described in Hoa, J. S. et al., as referenced above. Then, the PGS pyramid layer and Mg metal layer are laminated on POMaC. PLA spacer is used in between two inductor layers. PHB/PHV is used to encapsulate the film stack and sealed by applying pressure of 15 kPa and cut from sides of metal lines with 3 mm offset. Finally, the sensors are bent in between the capacitors to make it easy to mount during implantation and also make sure capacitors are placed on top and bottom of the artery. The assembled sensor is then peeled off from the wafer.

An example experimental pressure response and wireless measurement setup consists of a motorized vertical stage used in combination with a force gauge (digital force gauge series 5, Mark-10, USA). The capacitance of the sensor is measured with an E4980A Agilent Precision LCR meter. It is characterized at 15 kHz, frequency suitable for future miniaturized wireless custom-made readout circuit and far from 2.4 GHz and higher frequencies used in wireless network channels and various biomedical apparatus, to avoid any interference issue. Measurements are performed in controlled temperature and humidity atmosphere at 23±1° C. and 50±10% relative humidity. The wireless measurements are performed using a vector network analyzer (VNA) (E5071C Keysight, Agilent) using a custom-made reader coil (1 cm diameter and 1 mm wire width) and which is also employed to measure the S11 scattering parameter.

The following describes specific in vivo tests. For an example in vivo sensor function assessment, Sprague Dawley (SD) rats (300-350 g, male, ENVIGO) are used. The implantation surgery is performed under isoflurane inhalation anesthesia. The sensor is wrapped around the femoral vessel and fixed on the abductor muscles with sutures. For wireless sensor testing, the coil structure of the device is placed on the groin fat pad. Each animal is administered a dose enrofloxacin (Bayer Corp., Leverkusen, Germany) for antibiotic prophylaxis preoperatively and buprenorphine (Reckitt Benckiser Pharmaceuticals, Inc., Richmond, Va.) for pain control post-operatively. The rats are monitored throughout the study.

Biocompatibility of POMaC is evaluated histologically and compared to PGA and PLLA, which are FDA approved implantable medical materials. In brief, materials (n=3 for POMaC and n=2 for each PGA and PLLA (controls)) are implanted into subcutaneous pockets in upper backs of Sprague-Dawley rats (12-14 weeks, 300-350 g, male, ENVIGO). After three weeks, the materials are harvested with their surrounding soft tissue. The samples are then cut in half longitudinally; half for paraffin sections for H&E staining for evaluation of fibrous capsule formation surrounding the materials, and the other half for frozen sections for immunohistochemistry (IHC) for CD68, a surface marker of macrophages. The width of the fibrous capsule are measured at greater than five points per sample and the mean value is used for evaluation. For IHC analysis, at least five fields at ×10 magnification per section are selected at random within 1 mm of the implanted material on the superficial side. The number of CD68-positive cells in the fields is measured using ImageJ analysis software and the mean value is calculated per section (>5 fields/section in 5 sections for POMaC and >5 fields/section in 3 sections for each PGA and PLLA). All data are expressed as mean±standard deviation.

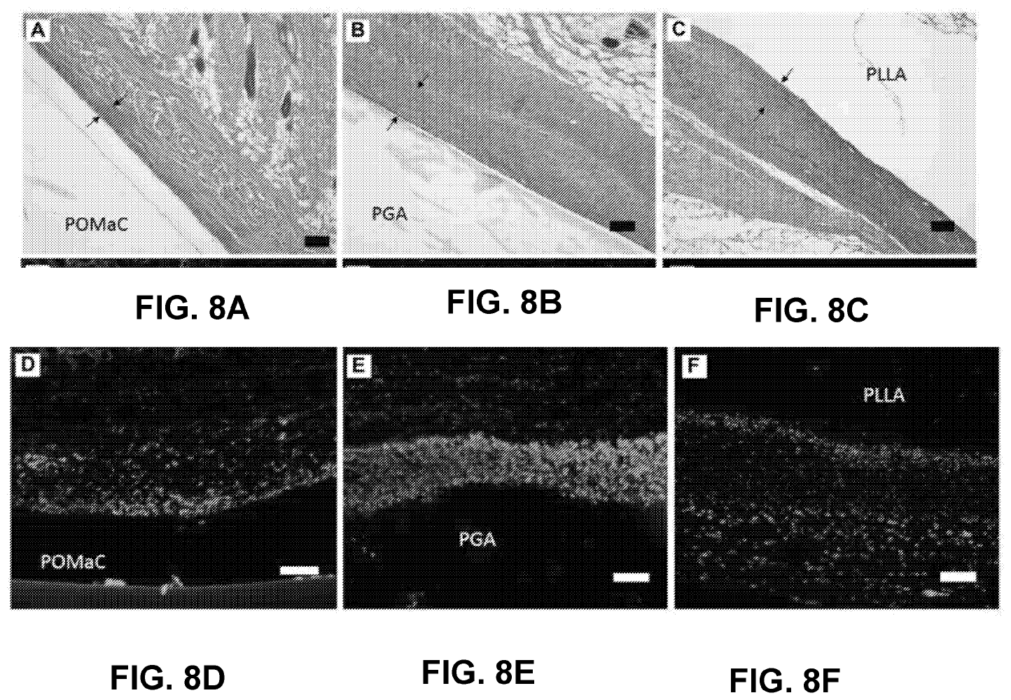

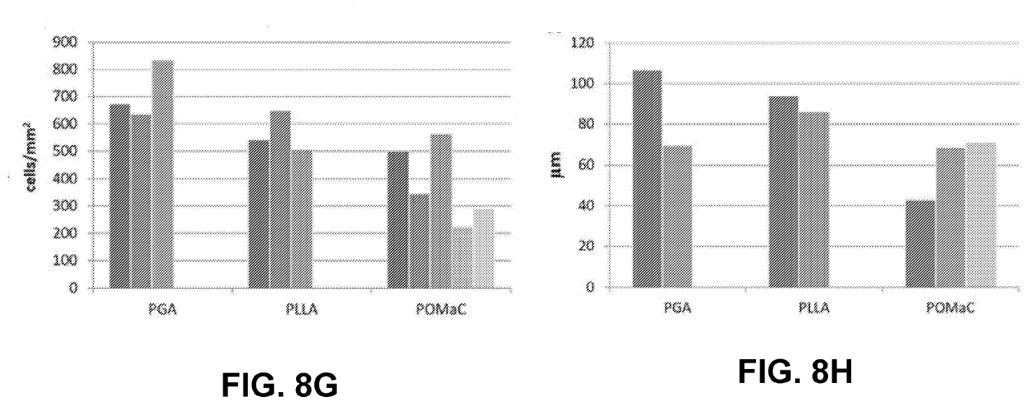

FIGS. 8A-8H show example results of an implanted sensor apparatus under different forces and resulting responses, consistent with embodiments of the present disclosure, as described above. The biocompatibility of POMaC during degradation in vivo is compared to PGA ((polyglycolic acid)) and PLLA (poly(lactic acid)). H&E staining is illustrated by the images of FIGS. 8A-8C and IHC for CD68 is illustrated by the images FIGS. 8D-8F at 3 weeks after implantation of POMaC (e.g., FIGS. 8A and 8D), PGA (e.g., FIGS. 8B and 8E), and PLLA (e.g., FIGS. 8C and 8F) showed fibrous capsular formation and significant number of CD68 positive cells around the materials. FIG. 8G illustrates quantification of the width of fibrous capsule and number of CD68+ cells per mm² 3 weeks after implantation. Each bar represents one section with a minimum of 5 representative images analyzed per section. FIG. 8H illustrates width of fibrous capsule 3 weeks after implantation. Each bar represents the average capsule width in one animal. A minimum of 5 measurements are taken per animal. Results reveal that POMaC has comparable biocompatibility to PGA and PLLA, which are FDA-approved medical implantable biomaterials. Scale bar: 100 μm

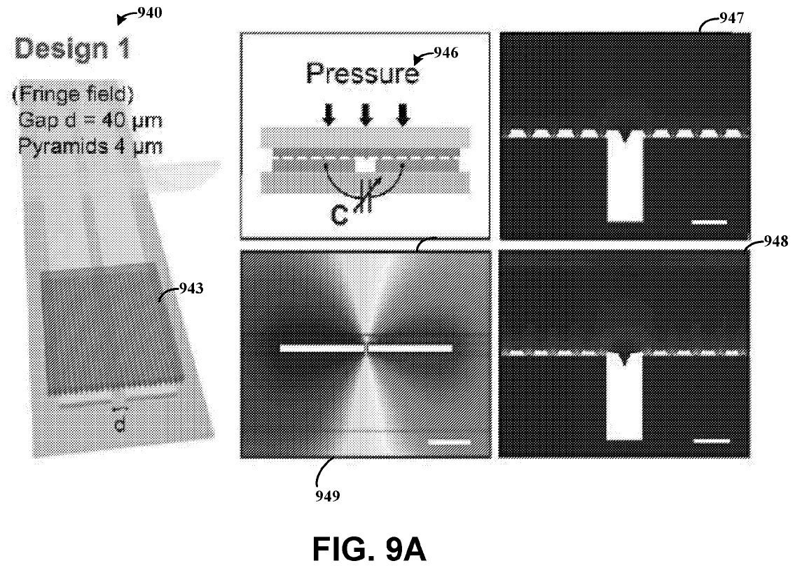

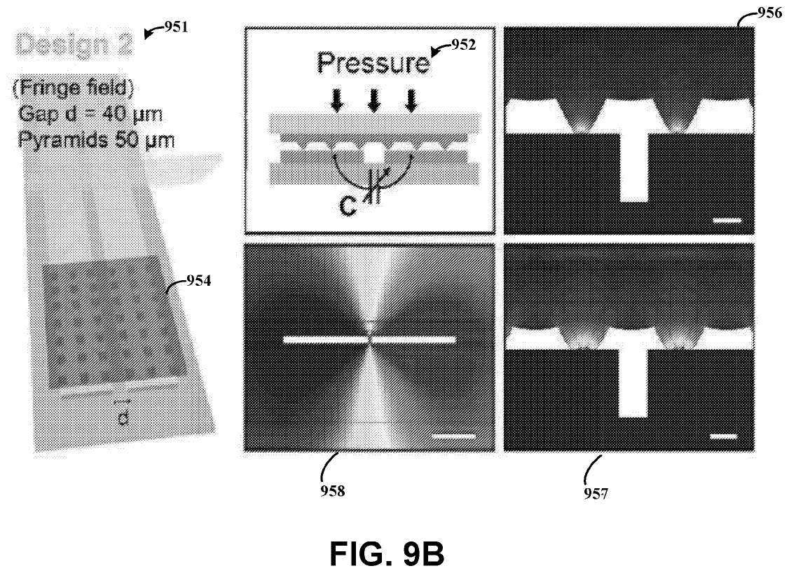

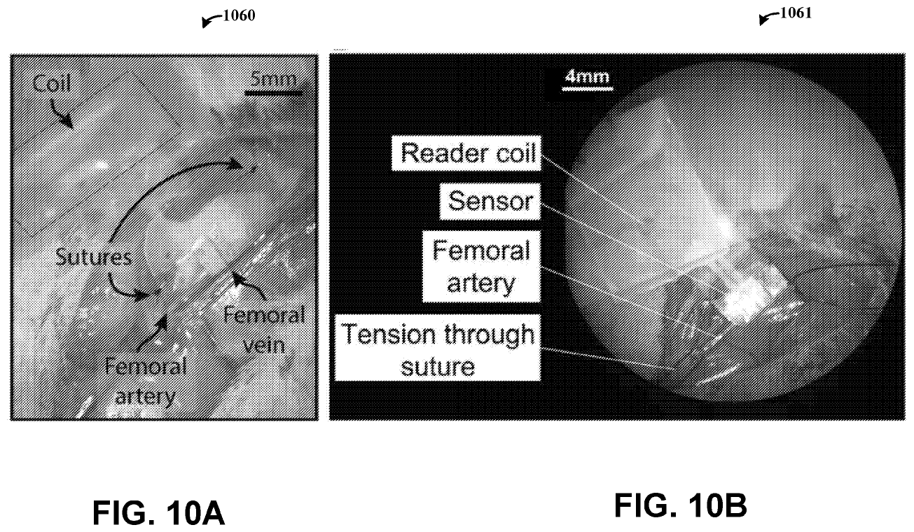

FIGS. 9A-9B show example performance of two different sensor apparatuses, consistent with embodiments of the present disclosure. More specifically, FIG. 9A illustrates design 1 and FIG. 9B illustrates design 2 as previously described in connection with FIG. 4A. Various experimental embodiments are directed to optimization fringe field capacitive sensor design. The fringe field capacitive sensors are evaluated with 2D finite element method (FEM) (Comsol simulation software). Multi-physics simulations are performed, including both mechanical and electrostatic models and a moving mesh, allowing for the evaluation of the mechanical deformation of the system under applied pressure, together with the corresponding capacitance change when mechanical deformation is applied, as shown by 947, 948, 956, and 957. As shown by FIGS. 9A-9B, design 1 and 2 for the fringe field capacitive sensor are evaluated with 2D finite element method (FEM) (Comsol simulation software), as shown by 949 and 958. In design 1, as shown by the top view 940 and side view 946 of FIG. 9A, the micro-structured elastomeric PGS layer 943 has small pyramids (base 4 μm, height 2.8 μm). In design 2, as shown by the top view 951 and side view 952 of FIG. 9B, the PGS layer 954 has large pyramids (base 50 μm, height 35.3 μm). The scale bar is 40 μm.

FIGS. 10A-10B show an example of an implanted sensor apparatus, consistent with embodiments of the present disclosure. More specifically, FIG. 10A is an image 1060 of an implanted sensor apparatus after suturing the sensor to femoral artery. FIG. 10B is an image 1061 of occlusion test design wherein two sutures are wrapped around the femoral artery distal and proximal to the sensor apparatus.

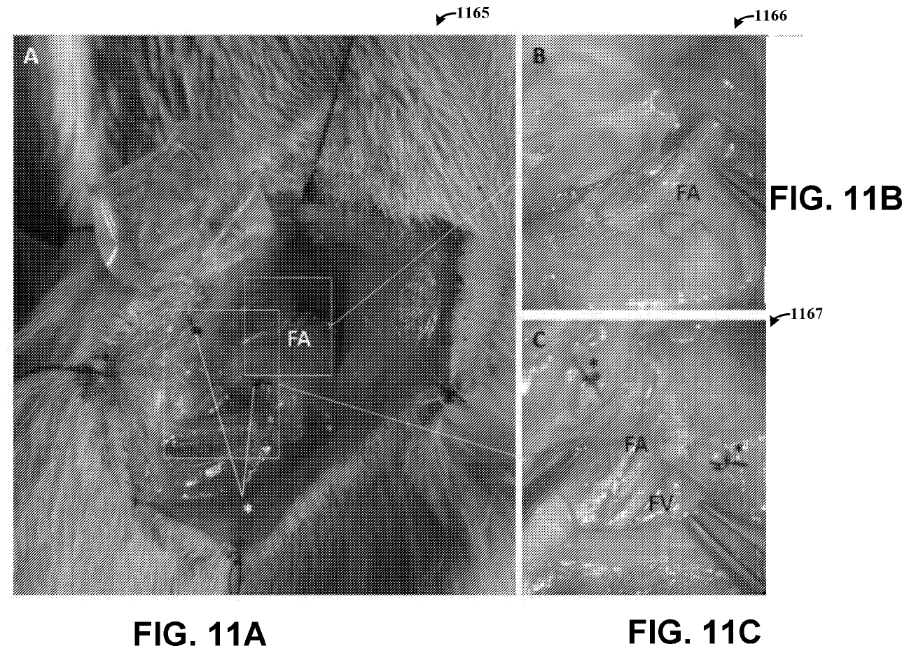

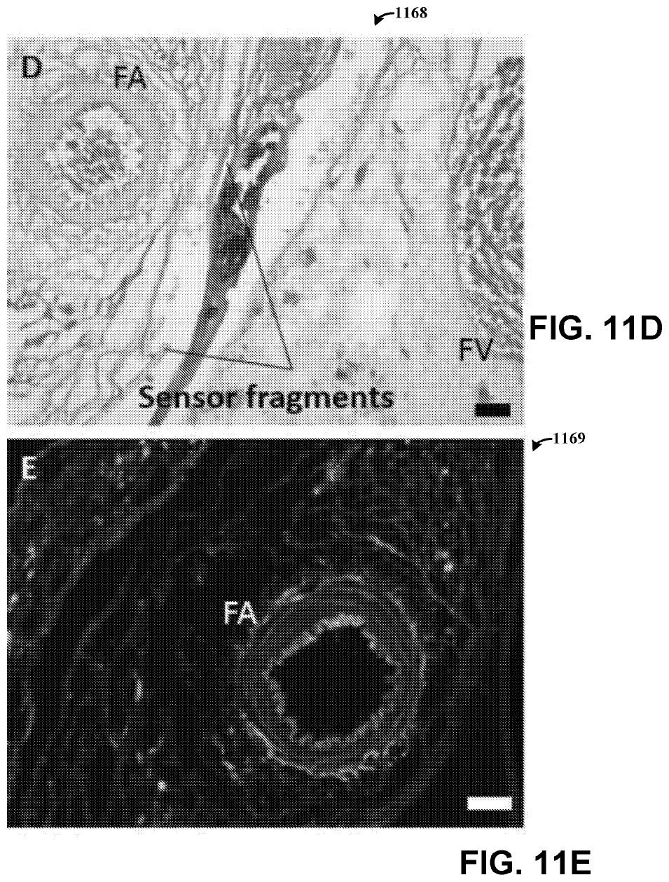

FIGS. 11A-11E show example of an implanted sensor apparatus, consistent with embodiments of the present disclosure. More specifically, FIGS. 11A-11E show example findings at three months after implantation of the sensor device. FIG. 11A is a macroscopic image 1165 and FIGS. 11B-11C are microscopic images 1166, 1167 at the harvest evidencing degradation of the enveloping material and almost complete absorption of the sensor and coil components. The sensor part and coil part are almost completely absorbed. Infra-superficial epigastric artery and vein are ligated and cut for exposure of the sensor and femoral artery, with FA (femoral artery) and FV (femoral vein). FIG. 11D illustrates an image 1168 of hematoxylin and eosin staining at three months with displayed red blood cells in the lumen of the femoral artery 3 months after implantation. Scale bar is 100 μm. FIG. 11E is an image 1169 of immunohistochemistry for CD68 at 3 months which reveals there is not a severe foreign body reaction around the femoral artery. Green is CD68, blue is DAPI, and the scale bar is 100 μm.

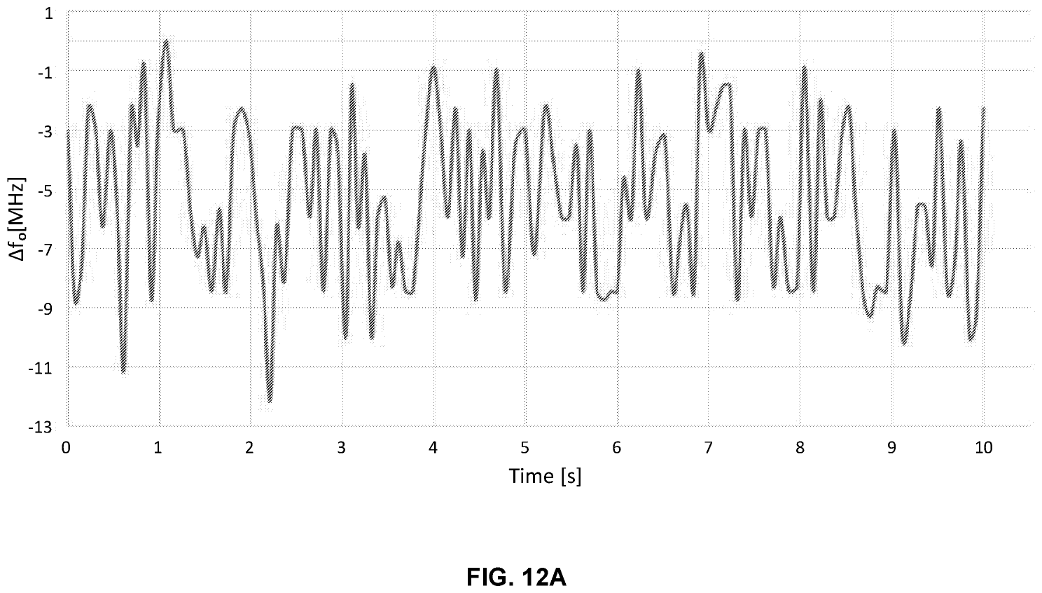

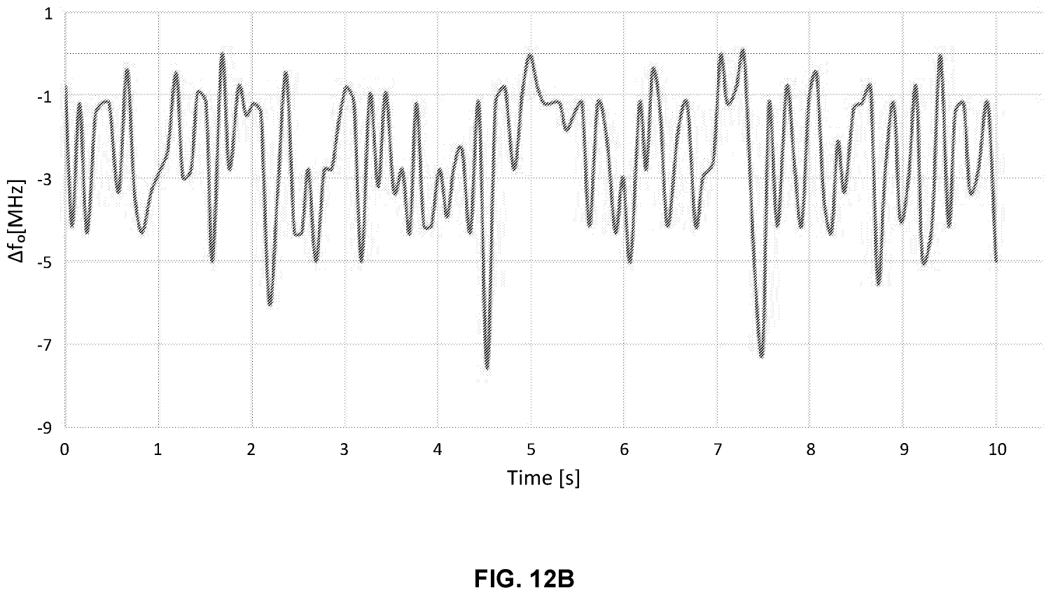

FIGS. 12A-12B show example resonant frequency shifts of a sensor apparatus, consistent with embodiments of the present disclosure. More specifically, FIGS. 12A-12B are graphs that show the resonant frequency shifts record during animal testing.

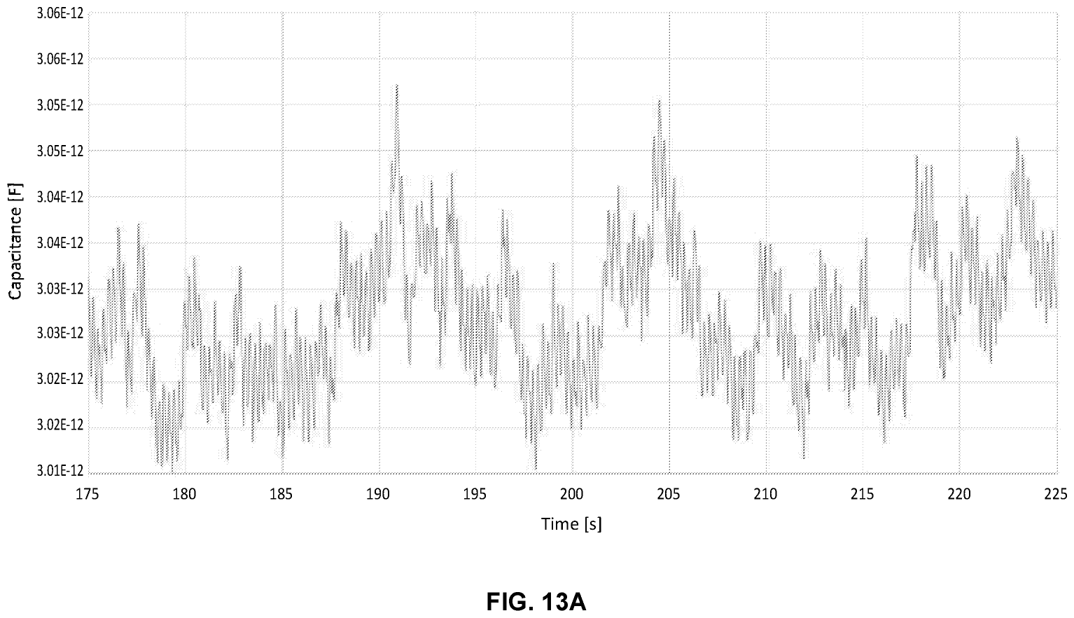

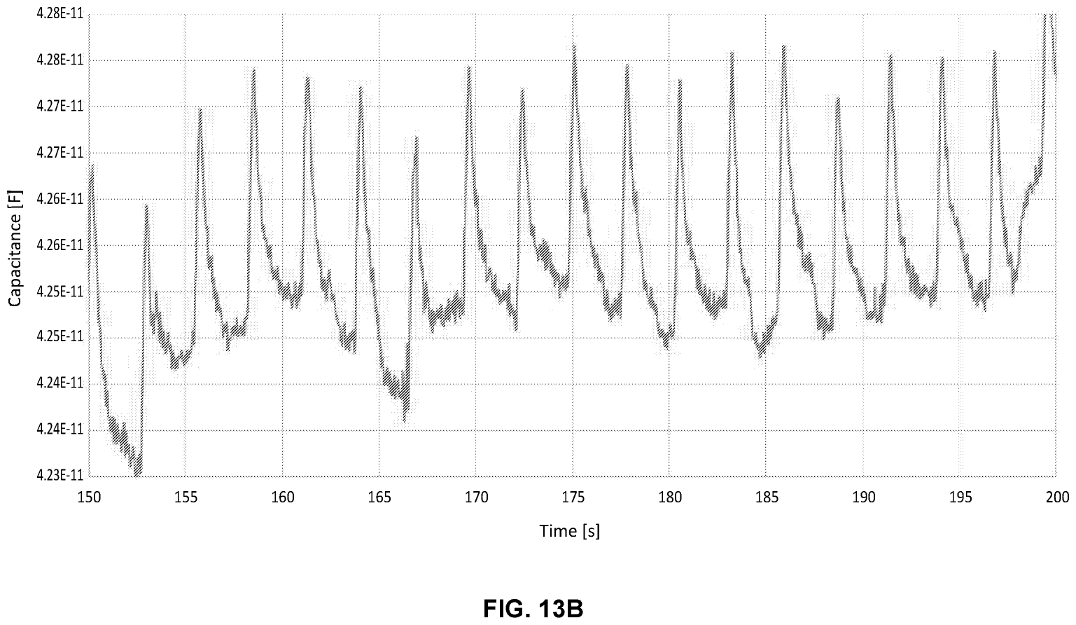

FIGS. 13A-13B show further example capacitance changes of a sensor apparatus, consistent with embodiments of the present disclosure. More specifically, FIGS. 13A-13B are graphs that show wired sensor in vivo characterization results with the proposed encapsulation method of POMaC and PHB/PHV illustrated by FIG. 13A and with both sides encapsulated with PHB/PHV illustrated by FIG. 13B. Softer film touching the artery while having a stiffer film on the side touching muscles increases sensor's sensitivity to artery expansion. On the other hand, having stiff films on both sides of the devices makes it more sensitive to respiratory motion instead of artery expansion.

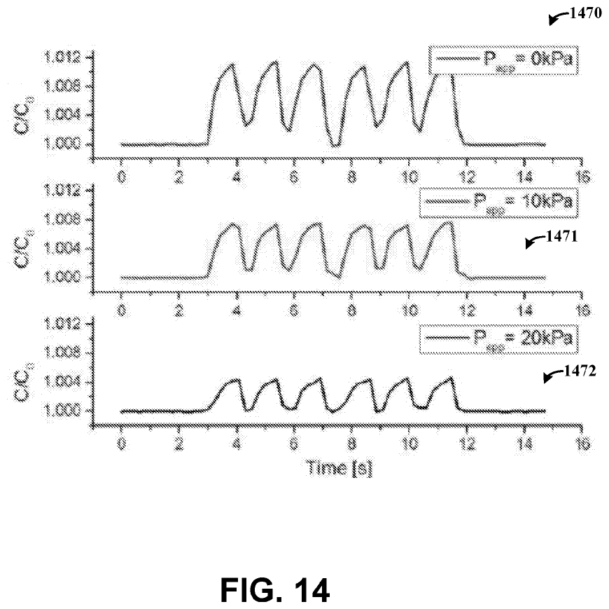

FIG. 14 shows example sensitivity of a sensor apparatus, consistent with embodiments of the present disclosure. Wired sensor in vitro characterization results with additional pressure applied to mimic effect of muscles and added pressures are illustrated by the graphs 1470, 1471, and 1472 which illustrated the sensitivity of the sensor decreases with increasing applied constant pressure.

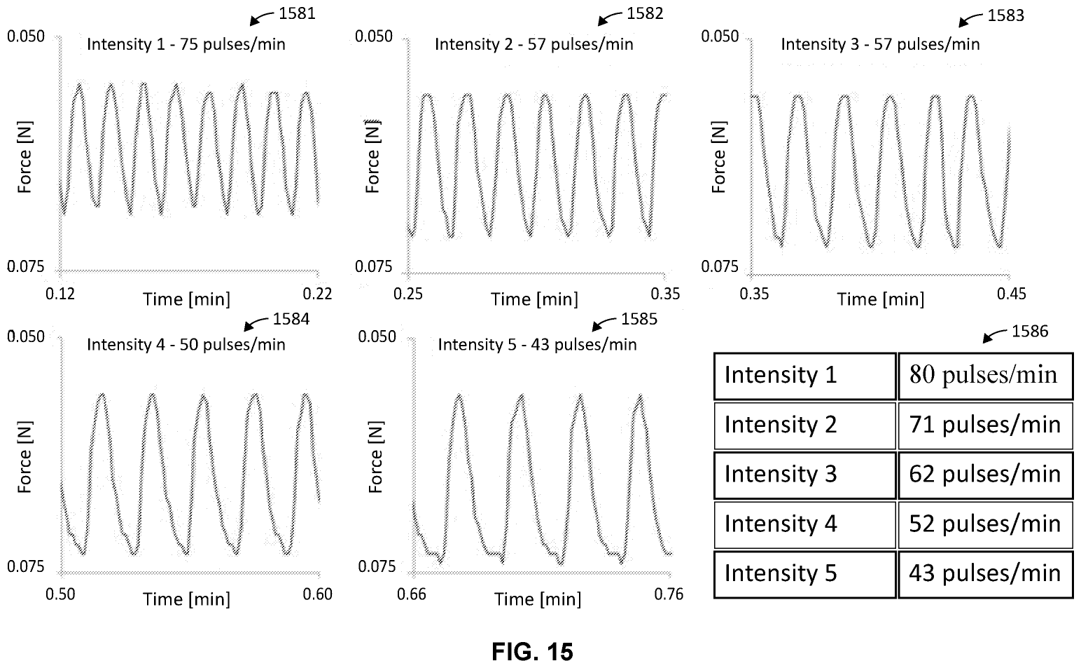

FIG. 15 shows example force characterized by a sensor apparatus, consistent with embodiments of the present disclosure. In vitro setup for artificial artery model is illustrated by supplementary FIG. S10 of the underlying provisional application. The setup includes a pressure measurement setup, and air pump (Philips Avent Electric Breast Pump SCF312/01) connected to the tube used as artificial artery model (in red, diameter 2 mm). The graphs 1581, 1582, 1583, 1584, 1585 illustrates the force is measured for pump settings 1 to 5 corresponding to 80 to 43 pulses per minute as shown by the table 1586 of FIG. 15. In specific examples, the young's modulus of the polymer materials used during fabrication of the sensor.

| Material name | Young's modulus | |
|---|---|---|
| POMaC | 0.5 | MPa |
| PLLA | 9.2 | GPa |
| PGS | 0.12 | MPa |
| PHB/PHV | 0.9 | MPa |

Various embodiments are implemented in accordance with the underlying Provisional Application (Ser. No. 62/750,518), entitled “Wireless Pulmonary Monitoring via Flexible Biodegradable Sensor Circuitry,” filed Oct. 25, 2018 which includes an Appendix entitled “Wireless Monitoring of Blood Flow via Biodegradable, Flexible, Passive Arterial Pulse Sensor,” and a Supplemental Appendix, entitled “Structural and Electrical Effect of Planar Double Capacitor Design” to which benefit is claimed and which are each fully incorporated herein by reference for their general and specific teachings. For instance, embodiments herein and/or in the Provisional Application and the Appendices be combined in varying degrees (including wholly). Reference may also be made to the experimental teachings and underlying references provided in the underlying Provisional Application. Embodiments discussed in the Provisional Application and Appendices are not intended, in any way, to be limiting to the overall technical disclosure, or to any part of the claimed disclosure unless specifically noted. The Provisional Application and Appendices illustrate a general sensor apparatus, and specific implementations of the sensor apparatus including the capacitive arrangement and the inductive arrangement, and experimental embodiments used to optimize the same. It is recognized that the various figures and descriptions herein can be used in combination with a variety of different structures and technical applications as described in the above-referenced Provisional Application and Appendices, which are fully incorporated herein by reference for all they contain.

Terms to exemplify orientation, such as top view/side view, before or after, upper/lower, left/right, top/bottom, and above/below, may be used herein to refer to relative positions of elements as shown in the figures. It should be understood that the terminology is used for notational convenience only and that in actual use the disclosed structures may be oriented differently than the orientation shown in the figures. Thus, the terms should not be construed in a limiting manner.

As examples, the Specification describes and/or illustrates aspects useful for implementing the claimed disclosure by way of various circuits or circuitry which may be illustrated as or using terms such as blocks, modules, device, system, unit, controller, and/or other circuit-type depictions. Such circuits or circuitry are used together with other elements (robotics, electronic devices, prosthetics, processing circuitry and the like) to exemplify how certain embodiments may be carried out in the form or structures, steps, functions, operations, activities, etc. For example, in certain of the above-discussed embodiments, one or more illustrated items in this context represent circuits (e.g., discrete logic circuitry or (semi)-programmable circuits) for implementing these operations/activities, as may be carried out in the approaches shown in the figures. In certain embodiments, such illustrated items represent one or more circuitry and/or processing circuitry (e.g., microcomputer or other CPU) which is understood to include memory circuitry that stores code (program to be executed as a set/sets of instructions) for performing a basic algorithm (e.g., inputting, counting signals having certain signal strength or amplitude, identifying pressure applied using changes in resonant frequency output by the sensor circuitry, sampling), and/or involving sliding window averaging, and/or a more complex process/algorithm as would be appreciated from known literature describing such specific-parameter sensing. Such processes/algorithms would be specifically implemented to perform the related steps, functions, operations, activities, as appropriate for the specific application.

Based upon the above discussion and illustrations, those skilled in the art will readily recognize that various modifications and changes may be made to the various embodiments without strictly following the exemplary embodiments and applications illustrated and described herein. For example, methods as exemplified in the Figures may involve steps carried out in various orders, with one or more aspects of the embodiments herein retained, or may involve fewer or more steps. Such modifications do not depart from the scope of various aspects of the disclosure, including aspects set forth in the claims.

What is claimed:

1. A sensor apparatus comprising:
two inductors with a first elastomer material between; and
at least one capacitor coupled to the two inductors, wherein the at least one capacitor is configured, while in use, to at least partially wrap a circumference of an object and to exhibit a change in impedance in response to a pressure-manifestation change associated with the object, the change in impedance is to cause a change in the resonant frequency of the two inductors.

2. The apparatus of claim 1, wherein the two inductors and the at least one capacitor include an inductance-capacitance-resistance (LCR) resonator circuit formed by first and second wires, and the two inductors are formed of portions of the first and second wires as respectively arranged in a coil.

3. The apparatus of claim 1, wherein the two inductors with the first elastomer material provide a wireless link to a reader coil currently with the least one capacitor exhibiting the change in impedance and causing the change in resonant frequency, wherein the wireless link provided is independent from sensing of the pressure-manifestation change.

4. The apparatus of claim 1, further including a second elastomer material proximal to a first of the two inductors and a third elastomer material proximal to the second of the two inductors, wherein the first, second, third elastomer materials are biodegradable.

5. The apparatus of claim 1, the at least one capacitor includes portions of first and second wires coupled to the two inductors, the portions of the first and second wires form first and second electrodes of the capacitor and have a dielectric material that expands the portions of the first and second wires, the dielectric material including a structured dielectric material that overlaps the portions of the first and second wires.

6. The apparatus of claim 1, wherein the at least one capacitor includes a fringe-field capacitor and the apparatus is biodegradable, and the sensor apparatus is configured to respond to pressure applied thereto in a contact mode and in response to a change in electromagnetic field in a non-contact mode.

7. The apparatus of claim 1, wherein the two inductors and the at least on capacitor are formed of a first wire and a second wire, and the at least one capacitor includes:
a first capacitor including a first portion of the first wire forming a first electrode and a first portion of the second wire forming a second electrode;
a second capacitor including a second portion of the first wire forming a third electrode and a second portion of the second wire forming a fourth electrode; and
a dielectric material including a first dielectric material expanding the first portions of the first and second wires and a second dielectric material expanding the second portions of the first and second wires.

8. The apparatus of claim 1, further including a reader coil and circuitry coupled to the reader coil to detect the change in the resonant frequency and to determine the pressure-manifestation change based on the change in the resonant frequency.

9. A sensor apparatus comprising:
a first inductive coil and a second inductive coil with a first elastomer material between;
a first wire coupled to a first inductive coil and a second wire coupled to the second inductive coil; and
a first capacitor including a first portion of the first wire and a first portion of the second wire, and a first dielectric material that expands between the first portions of the first and second wires, the first capacitor being configured to, while in use, at least partially wrap a circumference of an object and to exhibit a change in impedance in response to a pressure-manifestation change associated with the object, and the change in impedance is to cause a change in a resonant frequency of the first and second inductive coils.

10. The apparatus of claim 9, further including:
a second capacitor including a second portion of the first wire and a second portion of the second wire, and a second dielectric material that expands between the second portions of the first and second wires, wherein the first and second capacitors are configured to, while in use, to exhibit the change in impedance in response to the pressure-manifestation change associated with the object, and
a second elastomer material proximal to one of the first and second inductive coils and the first and second dielectric materials, and a third elastomer material proximal to the other of the first and second inductive coils.

11. The apparatus of claim 10, wherein the first and second capacitors are configured to wrap around a circular vessel and to exhibit the change in impedance in response to pressure applied by the circular vessel.

12. The apparatus of claim 9, wherein the first capacitor includes a fringe-field capacitor configured to wrap around an artery of a user and to exhibit the change in impedance in response to pressure applied or a change in electromagnetic field caused by the artery, and the sensor apparatus is biodegradable.

13. The apparatus of claim 9, wherein the first dielectric material includes a substrate with embedded three-dimensional (3D) microstructures.

14. The apparatus of claim 13, wherein the 3D microstructures include pyramid-shaped microstructures.

15. A method of forming a sensor apparatus comprising:
forming a first inductive coil coupled to a first wire and a second inductive coil coupled to a second wire from a conductive material;
forming a first elastomer material on one of the first and second inductive coils;
aligning the first inductive coil and the second inductive coil such that the first elastomer material is there between and the first and second wires extend from the first and second inductive coils at a first end of the first and second wires and with a distance between at a second end of the first and second wires distal to the first and second inductive coils; and
forming a dielectric material that expands a portion of the first and second wires proximal to the second ends of the first and second wires, wherein the portion of the first and second wires form at least one capacitor of the sensor apparatus, the at least one capacitor configured to, while in use, at least partially wrap a circumference of an object and to exhibit a change in impedance in response to a pressure-manifestation change associated with the object, and the change in impedance is to cause a change in a resonant frequency of the first and second inductive coils.

16. The method of claim 15, wherein forming the first and second inductive coils and the first and second wires includes laser cutting the first inductive coil coupled to the first wire and the second inductive coil coupled to the second wire from the conductive material, the first and second inductive coils being coupled together.

17. The method of claim 16, wherein aligning the first and second inductive coils includes folding the second inductive coil to align with the first inductive coil.

18. The method of claim 15, further including laminating the sensor apparatus with a second elastomer material proximal to one of the first and second inductive coils and the dielectric material and a third elastomer material proximal to the other of the first and second inductive coils.

19. The method of claim 15, wherein the first and second inductive coils form an antenna and the at least one capacitor forms a sensing region of the sensor apparatus.

20. The method of claim 15, wherein the formed sensor apparatus is biodegradable.
